(19) United States
(12) Patent Application Publication
(10) Pub. No.: US 2002/0119929 A1
(43) Pub. Date: Aug. 29, 2002

(54) Can1 and its role in mammalian infertility

(76) Inventors: Colin E. Bishop, Houston, TX (US); Alexander I. Agoulnik, Houston, TX (US); Qichao Zhu, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/003,806

(22) Filed: Nov. 2, 2001

Related U.S. Application Data
(60) Provisional application No. 60/245,872, filed on Nov. 3, 2000.

(51) Int. Cl. A61K 38/17; A61K 48/00

(52) U.S. Cl. 514/12; 514/44; 435/456; 435/458

(57) ABSTRACT

The present invention is directed to a Can1 mammalian sequence. Defects in this sequence result in aberrant migration and/or proliferation of primordial germ cells during embryonic development, leading to Sertoli Cell Only syndrome in males and Premature Ovarian Failure in females.

### FIELD OF THE INVENTION

The present invention is related to molecular and cellular biology, development, and reproductive biology. More specifically, the present invention is related to Can1 and its role in migration and/or proliferation of primordial germ cells during embryonic development.

### BACKGROUND OF THE INVENTION

In human populations, infertility is a common problem affecting 10-15% of all individuals. Defects in migration and/or proliferation of the primordial germ cells during embryonic development can lead to both Sertoli Cell Only syndrome (SCOS) in males and Premature Ovarian Failure (POF) in females, in which 0.3% of all young women are affected.

Premature ovarian failure (POF) in women is characterized as menopause that begins before the age of 35. Some cases of POF appear to be inherited. Pellas et al. (1991) originally isolated an insertional transgenic gcd (germ-cell deficient) mouse mutant having abnormal germ-cell development, and the gcd/gcd mouse is a useful animal model of premature ovarian failure in human females. The ged mutation is recessive and results in infertility in both males and females with no other detectable abnormalities in other tissues. More specifically, the germ cells were specifically depleted as early as day 11.5 of embryonic development, while the various somatic cells were apparently unaffected. Thus, the gcd locus plays an important role in the migration/proliferation of primordial germ cells to the genital ridges of developing embryos.

A study by Duncan et al. (1993) demonstrated that the gcd/gcd model animals closely mimic familial premature ovarian failure and were indeed useful for the characterization of the pathogenesis and treatment of POF. Shortly after puberty, the female gcd/gcd mice commenced reproductive senescence (indicated by high levels of circulating gonadotropins), the inability to respond to superovulation (either hormonally or functionally), and an interrupted estrous cycle. Other phenotypes include a complete absence of developing follicles in the ovaries and an inactive endometrium. However, the mice had normal age of vaginal opening, mammary gland histology, and sexual behavior, which suggests their sexual development was complete. Duncan and Chada (1993) demonstrated that the ovaries of young gcd/gcd mice are atrophic and comprise little more than a connective tissue matrix containing stromal cells. Following the first year of life, 56% of gcd/gcd mice develop tubulostromal adenoma, which also makes the gcd/gcd mice useful as a model for ovarian neoplasia.

In 1995, Duncan et al. mapped the mutation to mouse chromosome 11A2-3 using fluorescent in situ hybridization and DAPI chromosomal banding in conjunction with double labeling with the alpha 1(I) collagen gene. Although two candidate genes, Lif and Oncostatin M, map near the gcd locus, Southern blot hybridization analysis revealed no gross rearrangements in these genes in gcd mice, indicating these loci were not associated with the gcd phenotype.

### SUMMARY OF THE INVENTION

An embodiment of the present invention is a composition of matter of a nucleic acid sequence of SEQ ID NO: 1.

Another embodiment of the present invention is a composition of matter of an amino acid sequence of SEQ ID NO:3.

An additional embodiment of the present invention is a pharmaceutical composition comprising an amino acid sequence of SEQ ID NO:3; and a pharmacologically acceptable carrier.

Another embodiment of the present invention is a method of treating infertility in a mammal, comprising the step of introducing a therapeutically effective amount of a Can1 nucleic acid sequence into said mammal.

An additional embodiment of the present invention is a method of treating infertility in a mammal, comprising the step of introducing a therapeutically effective amount of a nucleic acid sequence of SEQ ID NO:1 into said mammal. In a specific embodiment, the nucleic acid sequence is introduced into said mammal in a vector. In another specific embodiment, the vector is selected from the group consisting of a plasmid, an adenovirus vector, an adeno-associated viral vector, a retroviral vector, a liposome, and a combination thereof.

Another embodiment of the present invention is a method of treating infertility in a mammal, comprising the step of introducing a therapeutically effective amount of a nucleic acid sequence of SEQ ID NO:2 into said mammal. In a specific embodiment, the nucleic acid sequence is introduced into said mammal in a vector. In an additional specific embodiment, the vector is selected from the group consisting of a plasmid, an adenovirus vector, an adeno-associated viral vector, a retroviral vector, a liposome, and a combination thereof.

An additional embodiment of the present invention is a method of treating infertility in a mammal comprising the step of introducing into said mammal a therapeutically effective amount of an amino acid sequence of SEQ ID NO:3.

A further embodiment of the present invention is a method of treating infertility in a mammal comprising the step of introducing to said mammal a therapeutically effective amount of an amino acid sequence of SEQ ID NO:4. In a specific embodiment, the amino acid sequence further comprises a protein transduction domain.

Another embodiment of the present invention is a method of diagnosing infertility in a mammal, comprising the steps of obtaining a biological sample from said mammal, wherein said sample includes a Can1 nucleic acid sequence; and assaying for a defect in said Can1 nucleic acid sequence. In a specific embodiment, the assaying step comprises an assay selected from the group consisting of polymerase chain reaction, nucleic acid hybridization, DNA chip analysis, sequencing, electrophoresis, and a combination thereof.

An additional embodiment of the present invention is a method of diagnosing infertility in a mammal, comprising the steps of obtaining a biological sample from said mammal, wherein said sample includes a Can1 amino acid sequence; and assaying for a defect said Can1 amino acid sequence. In a specific embodiment, the assaying step comprises an assay selected from the group consisting of mass spectrometry, sequencing, electrophoresis, immunoblot analysis, subcellular localization and a combination thereof.

An additional embodiment of the present invention is a method of increasing the number of primordial germ cells in a mammal, comprising the step of introducing into said mammal a physiologically significant level of a Can1 nucleic acid sequence.

Another embodiment of the present invention is a method of increasing the number of primordial germ cells in a mammal, comprising the step of introducing into said mammal a physiologically significant level of a Can1 amino acid sequence.

An additional embodiment of the present invention is a method of stimulating germ cell growth in a mammal, comprising the step of introducing into said mammal a physiologically effective level of a Can1 nucleic acid sequence.

An additional embodiment of the present invention is a method of stimulating germ cell growth in a mammal, comprising the step of introducing into said mammal a physiologically effective level of a Can1 amino acid sequence.

Another embodiment of the present invention is a method of screening for an active compound for the treatment of infertility, comprising the steps of obtaining an organism, wherein the genome of said organism includes a reporter sequence, wherein said reporter sequence expression is controlled by a Can1 regulatory nucleic acid sequence; exposing a test agent to said organism; and measuring a change in said expression, wherein said change indicates said test agent is said active compound. In a specific embodiment, the organism is a mouse. In another specific embodiment, the change in expression is an increase in expression.

An additional embodiment of the present invention is a method of screening in vitro for an active compound for the treatment of infertility, comprising the steps of obtaining a cell, wherein said cell includes a nucleic acid sequence having a reporter sequence, wherein said reporter sequence expression is controlled by a Can1 regulatory nucleic acid sequence; exposing a test agent to said cell; and measuring a change in said expression, wherein said change indicates said test agent is said active compound. In a specific embodiment, the cell is a mouse cell. In another specific embodiment, the reporter sequence is selected from the group consisting of β-galactosidase, β-glucuronidase, green fluorescent protein, blue fluorescent protein, and chloramphenicol acetyltransferase. In another specific embodiment, the change in said expression is an increase in said expression.

Another embodiment of the present invention is a method of screening for a candidate substance for the treatment of infertility comprising the steps of providing a cell lacking a functional Can1 amino acid sequence; contacting said cell with said candidate substance; and determining the effect of said candidate substance on said cell, wherein said effect on said cell is indicative said candidate substance treats said infertility.

An additional embodiment of the present invention is a transgenic non-human animal whose genome comprises a transgene encoding a Can1 amino acid sequence, wherein said transgene is under the control of an operably linked promoter active in eukaryotic cells. In a specific embodiment, the promoter is constitutive. In a specific embodiment, the promoter is tissue specific. In a specific embodiment, the promoter is inducible. In another specific embodiment, the animal is a mouse.

An additional embodiment of the present invention is a transgenic non-human animal comprising a genome with a heterozygous disruption of a nucleic acid encoding a Can1 polypeptide.

Another embodiment of the present invention is a transgenic non-human animal comprising a genome with a homozygous disruption of a nucleic acid encoding a Can1 polypeptide.

An additional embodiment of the present invention is a method of identifying an upregulator of Can1 nucleic acid sequence expression comprising the steps of administering a test compound to a transgenic non-human animal comprising a genome with a homozygous disruption of a nucleic acid encoding a Can1 polypeptide; measuring the level of said Can1 expression; and comparing the level of said Can1 expression in said animal with normal Can1 expression, wherein an increase in said level following administration of said test compound indicates said test compound is an upregulator.

Another embodiment of the present invention is a monoclonal antibody that binds immunologically to a polypeptide comprising SEQ ID NO:3, or an antigenic fragment thereof.

Another embodiment of the present invention is a polyclonal antisera, antibodies of which bind immunologically to a polypeptide comprising SEQ ID NO:3, or an antigenic fragment thereof.

An additional embodiment of the present invention is a monoclonal antibody that binds immunologically to a polypeptide comprising SEQ ID NO:4, or an antigenic fragment thereof.

An additional embodiment of the present invention is a polyclonal antisera, antibodies of which bind immunologically to a polypeptide comprising SEQ ID NO:4, or an antigenic fragment thereof.

Another embodiment of the present invention is a method of screening for an active compound for infertility, comprising the steps of introducing into a cell a first nucleic acid expressing a fused test peptide/DNA binding domain; and a second nucleic acid expressing a fused Can1 polypeptide/DNA activation domain; and assaying for an interaction between said test peptide and said Can1 polypeptide by measuring binding between said DNA binding domain and said DNA activation domain, wherein said interaction between said test peptide and said Can1 polypeptide indicates said test peptide is said active compound.

Another embodiment of the present invention is a method of treating an individual for premature ovarian failure, comprising the step of administering to said individual a nucleic acid sequence of SEQ ID NO:1.

An additional embodiment of the present invention is a method of treating an individual for premature ovarian failure, comprising the step of administering to said individual a nucleic acid sequence of SEQ ID NO:2.

Another embodiment of the present invention is a method of treating an individual for premature ovarian failure, comprising the step of administering to said individual an amino acid sequence of SEQ ID NO:3.

Another embodiment of the present invention is a method of treating an individual for premature ovarian failure, comprising the step of administering to said individual an amino acid sequence of SEQ ID NO:4.

An additional embodiment of the present invention is a method of treating an individual for Sertoli Cell only syndrome, comprising the step of administering to said individual a nucleic acid sequence of SEQ ID NO:1.

Another embodiment of the present invention is a method of treating an individual for Sertoli Cell only syndrome, comprising the step of administering to said individual a nucleic acid sequence of SEQ ID NO:2.

An additional embodiment of the present invention is a method of treating an individual for Sertoli Cell only syndrome, comprising the step of administering to said individual an amino acid sequence of SEQ ID NO:3.

Another embodiment of the present invention is a method of treating an individual for Sertoli Cell only syndrome, comprising the step of administering to said individual an amino acid sequence of SEQ ID NO:4.

Other and further objects, features and advantages would be apparent and eventually more readily understood by reading the following specification and by reference to the company drawing forming a part thereof, or any examples of the presently preferred embodiments of the invention are given for the purpose of the disclosure.

### BRIEF DESCRIPTION OF THE FIGURES

FIG. 1 illustrates histology sections of male gcd/+ (1A) and gcd/gcd testis (1B).

FIG. 2 illustrates histology sections of female gcd/+ (2A) and gcd/gcd ovaries (2B).

FIG. 3 shows in situ hybridization mapping of the transgene to chromosome 11 in a gcd/gcd mouse.

FIG. 4 demonstrates gel electrophoresis regarding somatic cell hybrid mapping of transgene insertion breakpoints to chromosome 11.

FIG. 5 shows a Southern analysis of transgene insertion breakpoints in gcd/gcd mice.

FIG. 6 illustrates mapping of gcd on BSS backcross panel.

FIG. 7 a schematic of the gcd transgene insertion site of chromosome 11.

FIG. 8 illustrates the Vrk2 and Can1 genes present in the gcd-deleted region of the gcd mouse.

FIG. 9 depicts the crossing strategy of the bacterial artificial chromosome (BAC) rescue using the Vrk2 gene.

FIG. 10 polymerase chain reaction/gel electrophoresis screening of transgenic mice in the Vrk2-BAC cross.

FIG. 11 demonstrates polymerase chain reaction/gel electrophoresis to identify gcd/gcd BAC transgenics using a Vrk2 polymorphism.

FIG. 12 demonstrates polymerase chain reaction/gel electrophoresis to demonstrate expression of Vrk2 BAC transgene in gonads of gcd/gcd mice.

FIG. 13 illustrates histology sections of testis of gcd/+ and gcd/gcd transgenic mice containing the Vrk2 BAC transgene.

FIG. 14 shows histology sections of ovary of gcd/+ and gcd/gcd transgenic mice containing the Vrk2 BAC transgene.

FIG. 15 demonstrates expression of Can1 in adult mouse testis using antisense probe (15A and 15B) and sense control probe (15C).

FIG. 16 illustrates testis histology of gcd/+ mice (16A) or gcd/knockout mice carrying the targeted Can1 gene.

FIG. 17 demonstrates testis histology of four week old gcd/knockout mice carrying the targeted Can1 gene.

FIG. 18 demonstrates ovarian histology of four week old Gcd/+ and gcd/knockout mice carrying the targeted Can1 gene.

### DESCRIPTION OF THE INVENTION

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “active compound” as used herein is a compound or agent which treats infertility.

The term “biological sample” as used herein is defined as a specimen from an individual for evaluation of a Can1 sequence and/or an fertility/infertility state. In a specific embodiment, the sample may be semen, ovarian tissue, testes tissue, urine, blood, saliva, sweat, tears, feces, skin or epithelial tissue, such as cheek scrapings, hair, or any part of a body of an individual.

The term “biologically functional equivalent” as used herein refers to a compound or biological entity which has functional activity similar to Can1. In a specific embodiment, the functional activity is the ability to cause primordial germ cells to migrate and/or proliferate during embryonic development. In another specific embodiment, the functional activity is the ability to cause an infertile individual to be fertile.

The term “enhancer” as used herein refers to a cis-acting regulatory sequence involved in the transcriptional activation of a nucleic acid sequence.

The term “exogenous” as used herein refers to a nucleic acid sequence which is foreign to a cell into which a vector is being introduced or that the sequence is homologous to a sequence in the cell but in a position within the host cell nucleic acid in which the sequence is ordinarily not found.

The term “expression vector” as used herein refers to a vector containing a nucleic acid sequence coding for at least part of a gene product capable of being transcribed. In a specific embodiment, the RNA molecules are then translated into a protein, polypeptide, or peptide. In another embodiment, these sequences are not translated, for example, in the production of antisense molecules or ribozymes.

The term “inducible elements” as used herein refers to regions of a nucleic acid sequence that can be activated in response to a specific stimulus.

The term “infertile” as used herein is defined as the inability to conceive or induce conception.

The term “mutant” as used herein refers to a change in the sequence of a nucleic acid or its encoded protein, polypeptide or peptide.

The terms “operatively positioned,” “operatively linked,” “under control,” and “under transcriptional control” as used herein mean that a promoter is in a correct functional location and/or orientation in relation to a nucleic acid sequence to control transcriptional initiation and/or expression of that sequence.

The term “polymorphic” as used herein means that variation exists (i.e. two or more alleles exist) at a genetic locus in the individuals of a population.

The term “premature ovarian failure” as used herein, also referred to as POF, is defined as secondary amenorrhea with elevated gonadotropins occurring before age 40. In a specific embodiment, POF also is characterized by early depletion of ova. In another specific embodiment, the condition is heterogeneous, and is present in individuals with chromosomal abnormalities (XO Turner), autoimmune problems, such as Addison's disease or myasthenia gravis, or galactosemia, 17α-hydroxylase deficiency and toxic damage. There is strong evidence that genetic components are associated with POF, such as BPES I syndrome (blepharophimosis, epicanthus inversus and ptosis), Chr. 3q22-23. In addition, mutations in the FSH receptor, on chromosome 2 also result in POF. Also, X-linked forms have been identified on Xq13-26, including mutations in DIA gene and microdeletions in FMR2 gene in fragile X cases. Approximately 32% of familial POF cases show autosomal recessive inheritance patterns.

The term “primer,” as used herein refers to any nucleic acid that is capable of priming the synthesis of a nascent nucleic acid in a template-dependent process.

The term “promoter” as used herein refers to a control sequence that is a region of a nucleic acid sequence at which initiation and rate of transcription are controlled. It may contain genetic elements at which regulatory proteins and molecules may bind such as RNA polymerase and other transcription factors. In a specific embodiment, a promoter may or may not be used in conjunction with an enhancer.

The term “Sertoli cell” as used herein is defined as an elongated cell in the testes tubuels to which spermatids become attached. Until the spermatids transform into mature spermatozoa, the Sertoli cells provide support, protection and nutrition to the spermatids. A skilled artisan is aware there are at least four types of Sertoli cells: 1) mature cells triangular in shape having an indented nucleus with a prominent tripartite nucleolus; 2) immature cells having an immature cytoplasm and round, regularly outlined nuclei; 3) poorly developed cells having immature nuclei and mostly mature cytoplasm having less developed organelles; and 4) involuting cells with mature cytoplasm having lipid droplets, residual bodies, and atypical inter-Sertoli junctional specializations and nuclei with irregular borders (Nistal et al., 1990).

The term “Sertoli cell only syndrome” as used herein is defined as a condition in males in which the testes contain solely Sertoli cells and are completely lacking in sperm of any stage of development. In a specific embodiment, the methods and compositions of the present invention are useful in therapy for individuals who have relatively few germ cells and are infertile.

The terms “treat,” “treats,” or “treatment” as used herein refer to action for resulting in an infertile individual becoming fertile.

The term “vector” as used herein refers to a carrier nucleic acid molecule into which a nucleic acid sequence can be inserted for introduction into a cell where it can be replicated.

The term “wild-type” as used herein refers to the naturally occurring sequence of a nucleic acid at a genetic locus in the genome of an organism, and sequences transcribed or translated from such a nucleic acid. In a specific embodiment, the term “wild-type” also refers to the amino acid sequence encoded by the nucleic acid. As a genetic locus may have more than one sequence or alleles in a population of individuals, the term “wild-type” encompasses all such naturally occurring alleles.

II. The Present Invention

Identification of genes that play a role in the early stages of primordial germ cell (PGC) development is key to the better understanding of conditions related to PGCs and their subsequent development. In these studies the non-pleiotropic, transgene-insertional, germ cell deficient mouse mutant (gcd) has been used as a model system. Analysis of this mutant clearly shows that disruption at a single locus can drastically reduce the number of primordial germ cells in the embryonic gonad by 11.5 days post-coitum (dpc), giving rise to male and female infertility in the adult. The male phenotype of a vacuolated testis, with only a few functional tubules, is very similar to the human Sertoli Cell Only syndrome (SCOS) seen in infertile males. The female phenotype of rapid follicular depletion closely parallels that of Premature Ovarian Failure. Thus, the gcd mouse is a useful model for investigating SCOS in males and POF in females.

In a specific embodiment, the following sequences are within the scope of the present invention and the corresponding GenBank Accession numbers are indicated where appropriate: mouse Can1 nucleic acid sequence (SEQ ID NO:1); mouse Can1 amino acid sequence (SEQ ID NO:3); human Can1 nucleic acid sequence ((SEQ ID NO:2; NM_{—}018062); (SEQ ID NO:5; AK001197); (SEQ ID NO:6; AC007250)); human Can1 amino acid sequence (SEQ ID NO:4; NP_{—}060532); Arabidopsis thaliana Can1 amino acid sequence (SEQ ID NO:7; BAB10681) and Drosophila melanogaster Can1 amino acid sequence (SEQ ID NO:8; AAF54486). Also within the scope of the present invention is the Can1 sequence the BAC clone RP11-334G22 (SEQ ID NO:9; AC007250) and the Can1 sequence of mouse chromosome 11 clone RP23-270L8 (SEQ ID NO:10; AC083815). A skilled artisan is aware of publicly available databases such as National Center Biotechnology Information's GenBank database or commercially available databases (Celera Genomics; Rockville, Md.) for retrieval of Can1 sequences or any sequences related to the present invention. A skilled artisan is also aware of public repositories for biological reagents such as cell lines, bacterial strains, media and the like, such as the American Type Culture Collection. In a specific embodiment, the 3′ untranslated region (UTR) of Can1 overlaps the 3′ UTR of another gene, such as Vrk2.

III. Nucleic Acid-Based Expression Systems

A. Vectors

Vectors include plasmids, cosmids, viruses (bacteriophage, animal viruses, and plant viruses), and artificial chromosomes (e.g., YACs or BACs). One of skill in the art would be well equipped to construct a vector through standard recombinant techniques, which are described in Maniatis et al., 1988 and Ausubel et al., 1994, both incorporated herein by reference.

Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

B. Promoters and Enhancers

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. Examples of promoters, enhancers, and inducible elements are well known in the art.

C. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

D. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

E. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

F. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

1. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

2. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

G. Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vectorencoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

H. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

IV. Nucleic Acid Detection

In addition to their use in directing the expression of Can1 proteins, polypeptides and/or peptides, the nucleic acid sequences disclosed herein have a variety of other uses. For example, they have utility as probes or primers for embodiments involving nucleic acid hybridization.

A. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, site-directed mutagenesis, it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KC1, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, calorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

Nucleic acids used as a template for amplification may be isolated from cells, tissues or other samples according to standard methodologies (Sambrook et al, 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

Typically, primers for polymerization are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to Can1 are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in their entirety.

A reverse transcriptase PCR™ amplification procedure may be performed to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et aL, 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). Davey et al., European Application No. 329 822 disclose a nucleic acid amplification process involving cyclically synthesizing single-stranded RNA (“ssRNA”), ssDNA, and double-stranded DNA (dsDNA), which may be used in accordance with the present invention.

Miller et al., PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art. See Sambrook et al, 1989. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR™ (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Patent No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

E. Kits

All the essential materials and/or reagents required for detecting Can1 in a sample may be assembled together in a kit. This generally will comprise a probe or primers designed to hybridize specifically to individual nucleic acids of interest in the practice of the present invention, including Can1. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally will comprise, in suitable means, distinct containers for each individual reagent or enzyme as well as for each probe or primer pair.

IV. Can1 Nucleic Acids

A. Nucleic Acids and Uses Thereof

An embodiment of the present invention concerns at least one Can1 nucleic acid. In a specific embodiment, the at least one Can1 nucleic acid comprises a wild-type or mutant Can1 nucleic acid. In another specific embodiment the at least one Can1 nucleic acid is the sequence complementary to the strand on which the Can1 sequence resides. In another specific embodiment, the Can1 nucleic acid comprises at least one transcribed nucleic acid. In other specific embodiments, the Can1 nucleic acid encodes at least one Can1 protein, polypeptide or peptide, or biologically functional equivalent thereof. In other specific embodiments, the Can1 nucleic acid comprises at least one nucleic acid segment of SEQ ID NO:1, SEQ ID NO:2, SEQ ID NO:5, SEQ ID NO:6, SEQ ID NO:9 or SEQ ID NO:10, or at least one biologically functional equivalent thereof.

One embodiment of the present invention is the isolation or creation of at least one recombinant construct or at least one recombinant host cell through the application of recombinant nucleic acid technology known to those of skill in the art or as described herein. The recombinant construct or host cell may comprise at least one Can1 nucleic acid, and may express at least one Can1 protein, peptide or peptide, or at least one biologically functional equivalent thereof.

A nucleic acid may be made by any technique known to one of ordinary skill in the art. Non-limiting examples of synthetic nucleic acid, particularly a synthetic oligonucleotide, include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986, and U.S. patent Ser. No. 5,705,629, each incorporated herein by reference. A non-limiting example of enzymatically produced nucleic acid include one produced by enzymes in amplification reactions such as PCR™ (see for example, U.S. Pat. Nos. 4,683,202 and 4,682,195, each incorporated herein by reference), or the synthesis of oligonucleotides described in U.S. Pat. No. 5,645,897, incorporated herein by reference. A non-limiting example of a biologically produced nucleic acid includes recombinant nucleic acid production in living cells, such as recombinant DNA vector production in bacteria (see for example, Sambrook et al. 1989, incorporated herein by reference).

A nucleic acid may be purified on polyacrylamide gels, cesium chloride centrifugation gradients, or by any other means known to one of ordinary skill in the art (see for example, Sambrook et al. 1989, incorporated herein by reference).

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Thus, the present invention also encompasses at least one nucleic acid that is complementary to a Can1 nucleic acid. In particular embodiments the invention encompasses at least one nucleic acid or nucleic acid segment complementary to the sequence set forth in SEQ ID NO:1, SEQ ID NO:2, SEQ ID NO:5, SEQ ID NO:6, SEQ ID NO:9 or SEQ ID NO:10. Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 75%, about 80%, about 85%, about 90%, about 95%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence of fornamide, tetramethylammonium chloride or other solvent(s) in the hybridization mixture. It is generally appreciated that conditions may be rendered more stringent, such as, for example, the addition of increasing amounts of formamide.

It is also understood that these ranges, compositions and conditions for hybridization are mentioned by way of non-limiting example only, and that the desired stringency for a particular hybridization reaction is often determined empirically by comparison to one or more positive or negative controls. Depending on the application envisioned it is preferred to employ varying conditions of hybridization to achieve varying degrees of selectivity of the nucleic acid(s) towards target sequence(s). In a non-limiting example, identification or isolation of related target nucleic acid(s) that do not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

One or more nucleic acid(s) may comprise, or be composed entirely of, at least one derivative or mimic of at least one nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refers to a molecule that may or may not structurally resemble a naturally occurring molecule, but functions similarly to the naturally occurring molecule. As used herein, a “moiety” generally refers to a smaller chemical or molecular component of a larger chemical or molecular structure, and is encompassed by the term “molecule.”

As used herein a “nucleobase” refers to a naturally occurring heterocyclic base, such as A, T, G, C or U (“naturally occurring nucleobase(s)”), found in at least one naturally occurring nucleic acid (i.e. DNA and RNA), and their naturally or non-naturally occurring derivatives and mimics. Non-limiting examples of nucleobases include purines and pyrimidines, as well as derivatives and mimics thereof, which generally can form one or more hydrogen bonds (“anneal” or “hybridize”) with at least one naturally occurring nucleobase in manner that may substitute for naturally occurring nucleobase pairing (e.g. the hydrogen bonding between A and T, G and C, and A and U).

Nucleobase, nucleoside and nucleotide mimics or derivatives are well known in the art, and have been described in exemplary references such as, for example, Scheit, Nucleotide Analogs (John Wiley, New York, 1980), incorporated herein by reference. “Purine” and “pyrimidine” nucleobases encompass naturally occurring purine and pyrimidine nucleobases and also derivatives and mimics thereof, including but not limited to, those purines and pyrimidines substituted by one or more of alkyl, carboxyalkyl, amino, hydroxyl, halogen (i.e. fluoro, chloro, bromo, or iodo), thiol, or alkylthiol wherein the alkyl group comprises of from about 1, about 2, about 3, about 4, about 5, to about 6 carbon atoms. Non-limiting examples of purines and pyrimidines include deazapurines, 2,6-diaminopurine, 5-fluoracil, xanthine, hypoxanthine, 8-bromoguanine, 8-chloroguanine, bromothymine, 8-aminoguanine, 8-hydroxyguanine, 8-methylguanine, 8-thioguanine, azaguanines, 2-aminopurine, 5-ethylcytosine, 5-methylcyosine, 5-bromouracil, 5-ethyluracil, 5-iodouracil, 5-chlorouracil, 5-propyluracil, thiouracil, 2-methyladenine, methylthioadenine, N,N-diemethyladenine, azaadenines, 8-bromoadenine, 8-hydroxyadenine, 6-hydroxyaminopurine, 6-thiopurine, 4-(6-aminohexyl/cytosine), and the like.

As used herein, “nucleoside” refers to an individual chemical unit comprising a nucleobase covalently attached to a nucleobase linker moiety. A non-limiting example of a “nucleobase linker moiety” is a sugar comprising 5-carbon atoms (a “5-carbon sugar”), including but not limited to deoxyribose, ribose or arabinose, and derivatives or mimics of 5-carbon sugars. Non-limiting examples of derivatives or mimics of 5-carbon sugars include 2′-fluoro-2′-deoxyribose or carbocyclic sugars where a carbon is substituted for the oxygen atom in the sugar ring. By way of non-limiting example, nucleosides comprising purine (i.e. A and G) or 7-deazapurine nucleobases typically covalently attach the 9 position of the purine or 7-deazapurine to the 1′-position of a 5-carbon sugar. In another non-limiting example, nucleosides comprising pyrimidine nucleobases (i.e. C, T or U) typically covalently attach the 1 position of the pyrimidine to 1′-position of a 5-carbon sugar (Kornberg and Baker, DNA Replication, 2nd Ed. (Freeman, San Francisco, 1992). However, other types of covalent attachments of a nucleobase to a nucleobase linker moiety are known in the art, and non-limiting examples are described herein.

As used herein, a “nucleotide” refers to a nucleoside further comprising a “backbone moiety” generally used for the covalent attachment of one or more nucleotides to another molecule or to each other to form one or more nucleic acids. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when the nucleotide comprises derivatives or mimics of a naturally occurring 5-carbon sugar or phosphorus moiety, and non-limiting examples are described herein.

A non-limiting example of a nucleic acid comprising such nucleoside or nucleotide derivatives and mimics is a “polyether nucleic acid”, described in U.S. patent Ser. No. 5,908,845, incorporated herein by reference, wherein one or more nucleobases are linked to chiral carbon atoms in a polyether backbone. Another example of a nucleic acid comprising nucleoside or nucleotide derivatives or mimics is a “peptide nucleic acid”, also known as a “PNA”, “peptide-based nucleic acid mimics” or “PENAMs”, described in U.S. patent Ser. Nos. 5,786,461, 5891,625, 5,773,571, 5,766,855, 5,736,336, 5,719,262, 5,714,331, 5,539,082, and WO 92/20702, each of which is incorporated herein by reference. A peptide nucleic acid generally comprises at least one nucleobase and at least one nucleobase linker moiety that is either not a 5-carbon sugar and/or at least one backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

Peptide nucleic acids may be utilized in embodiments of the present invention and generally have enhanced sequence specificity, binding properties, and resistance to enzymatic degradation in comparison to molecules such as DNA and RNA (Egholm et al., Nature 1993, 365, 566; PCT/EP/01219).

In certain aspects, the present invention concerns at least one nucleic acid that is an isolated nucleic acid. As used herein, the term “isolated nucleic acid” refers to at least one nucleic acid molecule that has been isolated free of, or is otherwise free of, the bulk of the total genomic and transcribed nucleic acids of one or more cells, particularly mammalian cells, and more particularly human and mouse cells. In certain embodiments, “isolated nucleic acid” refers to a nucleic acid that has been isolated free of, or is otherwise free of, bulk of cellular components and macromolecules such as lipids, proteins, small biological molecules, and the like. As different species may have a RNA or a DNA containing genome, the term “isolated nucleic acid” encompasses both the terms “isolated DNA” and “isolated RNA”. Thus, the isolated nucleic acid may comprise a RNA or DNA molecule isolated from, or otherwise free of, the bulk of total RNA, DNA or other nucleic acids of a particular species. As used herein, an isolated nucleic acid isolated from a particular species is referred to as a “species specific nucleic acid.” When designating a nucleic acid isolated from a particular species, such as human, such a type of nucleic acid may be identified by the name of the species. For example, a nucleic acid isolated from one or more humans would be an “isolated human nucleic acid”, a nucleic acid isolated from human would be an “isolated human nucleic acid”, etc.

Of course, more than one copy of an isolated nucleic acid may be isolated from biological material, or produced in vitro, using standard techniques that are known to those of skill in the art. In particular embodiments, the isolated nucleic acid is capable of expressing a protein, polypeptide or peptide that has Can1 activity. In other embodiments, the isolated nucleic acid comprises an isolated Can1 gene.

Herein certain embodiments, a “gene” refers to a nucleic acid that is transcribed. As used herein, a “gene segment” is a nucleic acid segment of a gene. In certain aspects, the gene includes regulatory sequences involved in transcription, or message production or composition. In particular embodiments, the gene comprises transcribed sequences that encode for a protein, polypeptide or peptide. In other particular aspects, the gene comprises a Can1 nucleic acid, and/or encodes a Can1 polypeptide or peptide coding sequences. In keeping with the terminology described herein, an “isolated gene” may comprise transcribed nucleic acid(s), regulatory sequences, coding sequences, or the like, isolated substantially away from other such sequences, such as other naturally occurring genes, regulatory sequences, polypeptide or peptide encoding sequences, etc. In this respect, the term “gene” is used for simplicity to refer to a nucleic acid comprising a nucleotide sequence that is transcribed, and the complement thereof. In particular aspects, the transcribed nucleotide sequence comprises at least one functional protein, polypeptide and/or peptide encoding unit. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Isolated substantially away from other coding sequences” means that the gene of interest, in this case the Can1 gene, forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

In certain embodiments, the nucleic acid is a nucleic acid segment. As used herein, the term “nucleic acid segment”, are smaller fragments of a nucleic acid, such as for non-limiting example, those that encode only part of the Can1 peptide or polypeptide sequence. Thus, a “nucleic acid segment” may comprise any part of the Can1 gene sequence(s), of from about 2 nucleotides to the full length of the Can1 peptide or polypeptide encoding region. In certain embodiments, the “nucleic acid segment” encompasses the full length Can1 gene(s) sequence. In particular embodiments, the nucleic acid comprises any part of the SEQ ID NO:1, SEQ ID NO:2, SEQ ID NO:5, SEQ ID NO:6, SEQ ID NO:9 or SEQ ID NO:10 sequence(s), of from about 2 nucleotides to the full length of the sequence disclosed in SEQ ID NO:1, SEQ ID NO:2, SEQ ID NO:5, SEQ ID NO:6, SEQ ID NO:9 or SEQ ID NO:10.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). The length overall length may vary considerably between nucleic acid constructs. Thus, a nucleic acid segment of almost any length may be employed, with the total length preferably being limited by the ease of preparation or use in the intended recombinant nucleic acid protocol.

In a non-limiting example, one or more nucleic acid constructs may be prepared that include a contiguous stretch of nucleotides identical to or complementary to SEQ ID NO:1, SEQ ID NO:2, SEQ ID NO:5, SEQ ID NO:6, SEQ ID NO:9 or SEQ ID NO:10. In particular embodiments, the invention concerns one or more recombinant vector(s) comprising nucleic acid sequences that encode a Can1 protein, polypeptide or peptide that includes within its amino acid sequence a contiguous amino acid sequence in accordance with, or essentially as set forth in SEQ ID NO:1, SEQ ID NO:2, SEQ ID NO:5, SEQ ID NO:6, SEQ ID NO:9 or SEQ ID NO:10. In other embodiments, the invention concerns recombinant vector(s) comprising nucleic acid sequences that encode a human Canl protein, polypeptide or peptide that includes within its amino acid sequence a contiguous amino acid sequence in accordance with, or essentially as set forth in SEQ ID NO:4. In particular aspects, the recombinant vectors are DNA vectors.

The term “a sequence essentially as set forth in SEQ ID NO:1, SEQ ID NO:2, SEQ ID NO:5, SEQ ID NO:6, SEQ ID NO:9 or SEQ ID NO:10 means that the sequence substantially corresponds to a portion of SEQ ID NO:3 or SEQ ID NO:4 and has relatively few amino acids that are not identical to, or a biologically functional equivalent of, the amino acids of SEQ ID NO:3 and/or SEQ ID NO:4. Thus, “a sequence essentially as set forth in SEQ ID NO:3” or “a sequence essentially as set forth in SEQ ID NO:4” encompasses nucleic acids, nucleic acid segments, and genes that comprise part or all of the nucleic acid sequences as set forth in SEQ ID NO:1, SEQ ID NO:2, SEQ ID NO:5, SEQ ID NO:6, SEQ ID NO:9 or SEQ ID NO:10.

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of amino acids that are identical or functionally equivalent to the amino acids of SEQ ID NO:3, SEQ ID NO:4, SEQ ID NO:7 or SEQ ID NO:8 will be a sequence that is “essentially as set forth in SEQ ID NO:3, SEQ ID NO:4, SEQ ID NO:7 or SEQ ID NO:8, provided the biological activity of the protein, polypeptide or peptide is maintained.

In certain other embodiments, the invention concerns at least one recombinant vector that include within its sequence a nucleic acid sequence essentially as set forth in SEQ ID NO:1, SEQ ID NO:2, SEQ ID NO:5, SEQ ID NO:6, SEQ ID NO:9 or SEQ ID NO:10. In particular embodiments, the recombinant vector comprises DNA sequences that encode protein(s), polypeptide(s) or peptide(s) exhibiting Can1 activity.

The term “functionally equivalent codon” is used herein to refer to codons that encode the same amino acid, such as the six codons for arginine and serine, and also refers to codons that encode biologically equivalent amino acids. The human DNA codons are well known in the art.

Information on codon usage in a variety of non-human organisms is known in the art (see for example, Bennetzen and Hall, 1982; Ikemura, 1981a, 1981b, 1982; Grantham et al., 1980, 1981; Wada et al., 1990; each of these references are incorporated herein by reference in their entirety). Thus, it is contemplated that codon usage may be optimized for other animals, as well as other organisms such as fungi, plants, prokaryotes, virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various noncoding sequences flanking either of the 5′ and/or 3′ portions of the coding region or may include various internal sequences, i.e., introns, which are known to occur within genes.

Excepting intronic and flanking regions, and allowing for the degeneracy of the genetic code, nucleic acid sequences that have between about 70% and about 79%; or more preferably, between about 80% and about 89%; or even more particularly, between about 90% and about 99%; of nucleotides that are identical to the nucleotides of SEQ ID NO:2, SEQ ID NO:5, SEQ ID NO:6 or SEQ ID NO:9 will be nucleic acid sequences that are “essentially as set forth in SEQ ID NO:2, SEQ ID NO:5, SEQ ID NO:6 or SEQ ID NO:9”.

It will also be understood that this invention is not limited to the particular nucleic acid or amino acid sequences of SEQ ID NO:3, SEQ ID NO:4, SEQ ID NO:7, or SEQ ID NO:8. Recombinant vectors and isolated nucleic acid segments may therefore variously include these coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present invention encompass biologically functional equivalent Can1 proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine Can1 protein, polypeptide or peptide activity at the molecular level.

Fusion proteins, polypeptides or peptides may be prepared, e.g., where the Can1 coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding relatively small peptides or fusion peptides, such as, for example, peptides of from about 5, about 10, about 15, about 20, about 25, about 30, about 35, about 40, about 45, about 50, about 55, about 60, about 65, about 70, about 75, about 80, about 85, about 90, about 95, to about 100 amino acids in length, or more preferably, of from about 15 to about 30 amino acids in length; as set forth in SEQ ID NO:3, SEQ ID NO:4, SEQ ID NO:7 or SEQ ID NO:8 and also larger polypeptides up to and including proteins corresponding to the full-length sequences set forth in SEQ ID NO:3, SEQ ID NO:4, SEQ ID NO:7 or SEQ ID NO:8.

As used herein an “organism” may be a prokaryote, eukaryote, virus and the like. As used herein the term “sequence” encompasses both the terms “nucleic acid” and “proteinaceous” or “proteinaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e. an “enzymatically produced” sequence) or biological production in vivo (i.e. a “biologically produced” sequence).

V. Pharmaceutical Compositions and Pharmaceutically Acceptable Carriers

Aqueous compositions of the present invention comprise an effective amount of a Can1 protein, polypeptide, peptide, epitopic core region, inhibitor, and/or such like, dissolved and/or dispersed in a pharmaceutically acceptable carrier and/or aqueous medium. Aqueous compositions of gene therapy vectors expressing any of the foregoing are also contemplated.

The phrases “pharmaceutically and/or pharmacologically acceptable” refer to molecular entities and/or compositions that do not produce an adverse, allergic and/or other untoward reaction when administered to an animal as appropriate.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety and/or purity standards as required by FDA Office of Biologics standards.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of a Can1 agent as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

Can1 protein, polypeptide, peptide, agonist and/or antagonist of the present invention can be formulated into a composition in a neutral and/or salt form. Pharmaceutically acceptable salts, include the acid addition salts (formed with the free amino groups of the protein) and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof. The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 ml of isotonic NaCl solution and/or either added to 1000 ml of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

The Can1 protein-derived peptides and/or agents may be formulated within a therapeutic mixture to comprise about 0.0001 to 1.0 milligrams, and/or about 0.001 to 0.1 milligrams, and/or about 0.1 to 1.0 and/or even about 10 milligrams per dose and/or so. Multiple doses can also be administered.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation. Various commercial nasal preparations are known and/or include, for example, antibiotics and/or antihistamines and/or are used for asthma prophylaxis.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

VI. Lipid Formulations and/or Nanocapsules

In certain embodiments, the use of lipid formulations and/or nanocapsules is contemplated for the introduction of Can1 protein, polypeptides, peptides and/or agents, and/or gene therapy vectors, including both wild-type and/or antisense vectors, into host cells.

Nanocapsules can generally entrap compounds in a stable and/or reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and/or such particles may be easily made.

In a preferred embodiment of the invention, the Can1 may be associated with a lipid. The Can1 associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The lipid or lipid/Can1 associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes.

Phospholipids may be used for preparing the liposomes according to the present invention and may carry a net positive, negative, or neutral charge. Diacetyl phosphate can be employed to confer a negative charge on the liposomes, and stearylamine can be used to confer a positive charge on the liposomes. The liposomes can be made of one or more phospholipids.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Phospholipids can form a variety of structures other than liposomes when dispersed in water, depending on the molar ratio of lipid to water. At low ratios the liposome is the preferred structure. The physical characteristics of liposomes depend on pH, ionic strength and/or the presence of divalent cations. Liposomes can show low permeability to ionic and/or polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and/or results in an increase in permeability to ions, sugars and/or drugs.

Liposomes interact with cells via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Liposome-mediated oligonucleotide delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the lipid may be associated with a hemagglutinating virus (HVJ). This has been shown to facilitate fusion with the cell membrane and promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1. In that such expression vectors have been successfully employed in transfer and expression of an oligonucleotide in vitro and in vivo, then they are applicable for the present invention. Where a bacterial promoter is employed in the DNA construct, it also will be desirable to include within the liposome an appropriate bacterial polymerase.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one preferred embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000× g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

A pharmaceutical composition comprising the liposomes will usually include a sterile, pharmaceutically acceptable carrier or diluent, such as water or saline solution.

VII. Kits

Therapeutic kits of the present invention are kits comprising Can1 protein, polypeptide, peptide, inhibitor, gene, vector and/or other Can1 effector. Such kits will generally contain, in suitable container means, a pharmaceutically acceptable formulation of Can1 protein, polypeptide, peptide, domain, inhibitor, and/or a gene and/or vector expressing any of the foregoing in a pharmaceutically acceptable formulation. The kit may have a single container means, and/or it may have distinct container means for each compound.

When the components of the kit are provided in one and/or more liquid solutions, the liquid solution is an aqueous solution, with a sterile aqueous solution being particularly preferred. The Can1 compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the Can1 protein, gene and/or inhibitory formulation are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate Can1 protein and/or gene composition within the body of an animal. Such an instrument may be a syringe, pipette, forceps, and/or any such medically approved delivery vehicle.

VIII. Therapeutically Effective Level

As used in the present invention, a compound is therapeutically effective if it decreases, delays or eliminates the onset of infertility or if it decreases, delays or improves any symptom associated with infertility. A skilled artisan readily recognizes that in many of these cases the compound may not provide a cure but may only provide partial benefit. A physiological change having some benefit is considered therapeutically beneficial. Thus, an amount of compound which provides a physiological change is considered an “effective amount” or a therapeutically effective amount.”

A compound, molecule or composition is said to be “pharmacologically acceptable” if its administration can be tolerated by a recipient mammal. Such an agent is said to be administered in a “therapeutically effective amount” if the amount administered is physiologically significant. An agent is physiologically significant if its presence results in technical change in the physiology of a recipient mammal. For example, in the treatment of infertility of the present invention, a compound is therapeutically effective if it (i) results in fertility for a previously infertile individual; or (2) delays onset of symptoms of infertility.

IX. Kits

Therapeutic kits of the present invention are kits comprising Can1 protein, polypeptide, peptide, inhibitor, gene, vector and/or other Can1 effector. Such kits generally contain, in suitable container means, a pharmaceutically acceptable formulation of Can1 or any Can1 protein, polypeptide, peptide, domain, inhibitor, and/or a gene and/or vector expressing any of the foregoing in a pharmaceutically acceptable formulation. The kit may have a single container means, and/or it may have distinct container means for each compound.

When the components of the kit are provided in one and/or more liquid solutions, the liquid solution is an aqueous solution, with a sterile aqueous solution being particularly preferred. The Can1 protein, polypeptide, peptide, domain, inhibitor, or effector compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means generally includes at least one vial, test tube, flask, bottle, syringe and/or other container means, into which Can1 protein, polypeptide, peptide, domain, inhibitor, or effector formulation are placed, preferably, suitably allocated. The kits may also comprise a second container means for containing a sterile, pharmaceutically acceptable buffer and/or other diluent.

The kits of the present invention also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate Can1 protein, polypeptide, peptide, domain, inhibitor, or effector within the body of an animal. Such an instrument may be a syringe, pipette, forceps, and/or any such medically approved delivery vehicle.

X. Gene Therapy Administration

For gene therapy, a skilled artisan would be cognizant that the vector to be utilized must contain the gene of interest operatively limited to a promoter. For antisense gene therapy, the antisense sequence of the gene of interest would be operatively linked to a promoter. For promoter therapy, specific NFκB repressor or enhancer sites are introduced as contiguous DNA double-stranded molecules with weak promoter properties. This kind of therapy effects native transcription factors in the cell, namely NFκB and/or ETS. In this embodiment, there is no gene expression from the repressor or enhancer sites within the vector, but they are effective in titrating endogenous NFKB and/or ETS transcription factors away from normal functioning sites. One skilled in the art recognizes that in certain instances other sequences such as a 3′ UTR regulatory sequences are useful in expressing the gene of interest. Where appropriate, the gene therapy vectors can be formulated into preparations in solid, semisolid, liquid or gaseous forms in the ways known in the art for their respective route of administration. Means known in the art can be utilized to prevent release and absorption of the composition until it reaches the target organ or to ensure timed-release of the composition. A pharmaceutically acceptable form should be employed which does not ineffectuate the compositions of the present invention. In pharmaceutical dosage forms, the compositions can be used alone or in appropriate association, as well as in combination, with other pharmaceutically active compounds. A sufficient amount of vector containing the therapeutic nucleic acid sequence must be administered to provide a pharmacologically effective dose of the gene product.

One skilled in the art recognizes that different methods of delivery may be utilized to administer a vector into a cell. Examples include: (1) methods utilizing physical means, such as electroporation (electricity), a gene gun (physical force) or applying large volumes of a liquid (pressure); and (2) methods wherein said vector is complexed to another entity, such as a liposome or transporter molecule.

Accordingly, the present invention provides a method of transferring a therapeutic gene to a host, which comprises administering the vector of the present invention, preferably as part of a composition, using any of the aforementioned routes of administration or alternative routes known to those skilled in the art and appropriate for a particular application. Effective gene transfer of a vector to a host cell in accordance with the present invention to a host cell can be monitored in terms of a therapeutic effect (e.g. alleviation of some symptom associated with the particular disease being treated) or, further, by evidence of the transferred gene or expression of the gene within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer).

These methods described herein are by no means all-inclusive, and further methods to suit the specific application are apparent to the ordinary skilled artisan. Moreover, the effective amount of the compositions can be further approximated through analogy to compounds known to exert the desired effect.

Furthermore, the actual dose and schedule can vary depending on whether the compositions are administered in combination with other pharmaceutical compositions, or depending on interindividual differences in pharmacokinetics, drug disposition, and metabolism. Similarly, amounts can vary in in vitro applications depending on the particular cell line utilized (e.g., based on the number of vector receptors present on the cell surface, or the ability of the particular vector employed for gene transfer to replicate in that cell line). Furthermore, the amount of vector to be added per cell likely vary with the length and stability of the therapeutic gene inserted in the vector, as well as also the nature of the sequence, and is particularly a parameter which needs to be determined empirically, and can be altered due to factors not inherent to the methods of the present invention (for instance, the cost associated with synthesis). One skilled in the art can easily make any necessary adjustments in accordance with the exigencies of the particular situation.

It is possible that cells containing the therapeutic gene may also contain a suicide gene (i.e., a gene which encodes a product that can be used to destroy the cell, such as herpes simplex virus thymidine kinase). In many gene therapy situations, it is desirable to be able to express a gene for therapeutic purposes in a host cell but also to have the capacity to destroy the host cell once the therapy is completed, becomes uncontrollable, or does not lead to a predictable or desirable result. Thus, expression of the therapeutic gene in a host cell can be driven by a promoter although the product of said suicide gene remains harmless in the absence of a prodrug. Once the therapy is complete or no longer desired or needed, administration of a prodrug causes the suicide gene product to become lethal to the cell. Examples of suicide gene/prodrug combinations which may be used are Herpes Simplex Virus-thymidine kinase (HSV-tk) and ganciclovir, acyclovir or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The method of cell therapy may be employed by methods known in the art wherein a cultured cell containing a copy of a nucleic acid sequence or amino acid sequence of a sequence of interest is introduced.

XI. Methods of Making Transgenic Mice

A particular embodiment of the present invention provides transgenic animals that contain Can1-related constructs. Transgenic animals expressing Can1, recombinant cell lines derived from such animals, and transgenic embryos may be useful in methods for screening for and identifying agents that interact with Can1, or affect infertility through utilization of Can1. The use of constitutively-expressed Can1 provides a model for over- or unregulated expression, compared to normal basal expression levels. Also, transgenic animals which are “knocked out” for Can1 are utilized, such as for screening methods or as models for therapeutic assays for candidate compounds.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), Brinster et al. 1985; which is incorporated herein by reference in its entirety) and in “Manipulating the Mouse Embryo; A Laboratory Manual” 2nd edition (eds., Hogan, Beddington, Costantimi and Long, Cold Spring Harbor Laboratory Press, 1994; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM TrispH 7.4, and 1 mM EDTA) and purified on an Elutip-D™Mcolumn. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA.

Other methods for purification of DNA for microinjection are described in Hogan et al. Manipulating the Mouse Embryo (Cold Spring Harbor Laboratory, Cold Spring Harbor, N.Y., 1986), in Palmiter et al. Nature 300:611 (1982); in The Qiagenologist, Application Protocols, 3rd edition, published by Qiagen, Inc., Chatsworth, Calif.; and in Sambrook et al Molecular Cloning: A Laboratory Manual (Cold Spring Harbor Laboratory, Cold Spring Harbor, N.Y., 1989), all of which are incorporated by reference herein.

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by C0₂ asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. FVB, C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

A skilled artisan is aware that transgenic mice are also commercially available, such as from Charles River Laboratories (Wilmington, Mass.).

XII. Zinc Finger Proteins

In a specific embodiment, Canl protein, polypeptide or peptide comprises a zinc finger region (for review, see Leon and Roth, 2000). In a further specific embodiment, Can1 comprises a cysteine-rich RING-H2 finger motif. Ring finger domains, through their action as scaffolds for building multiprotein complexes, have been implicated in multiple cellular processes including ubiquitination, transcription, signal transduction, and RNA transport (for review, see Saurin et al., 1996).

### EXAMPLES

The following examples are offered by way of example, and are not intended to limit the scope of the invention in any manner.

### Example 1

### gcd/gcd Male Infertility is Due to Severe Oligospermia Progressing to Azoospermia

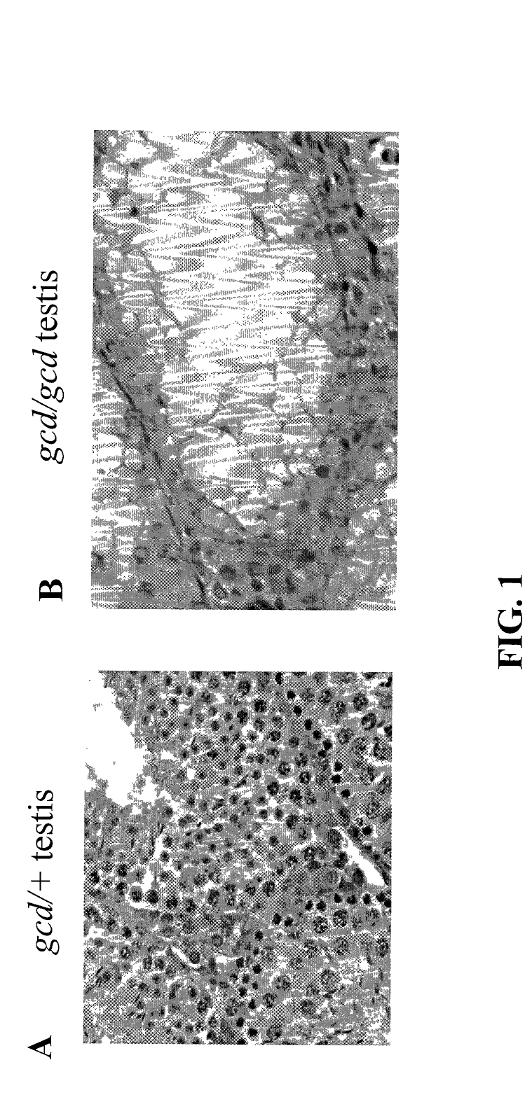

To characterize cellular morphology in gcd/gcd male mutants, which mimic the phenotypes of Sertoli Cell Only Syndrome in human males, testis were extracted from the gcd/gcd mutants or littermate gcd/+ controls, sectioned, and stained with standards reagents known in the art. FIGS. 1A and 1B illustrate abnormal testis morphology in gcd/gcd mutants, compared to gcd/+ heterozygote testis. Abnormal semeniferous tubules with vacuolated Sertoli cells are hallmarks of gcd/gcd testis, which is approximately one-third normal size.

### Example 2

### Female Infertility is Due to Lack of Developing Follicles and Rapid Atresia of Oocytes

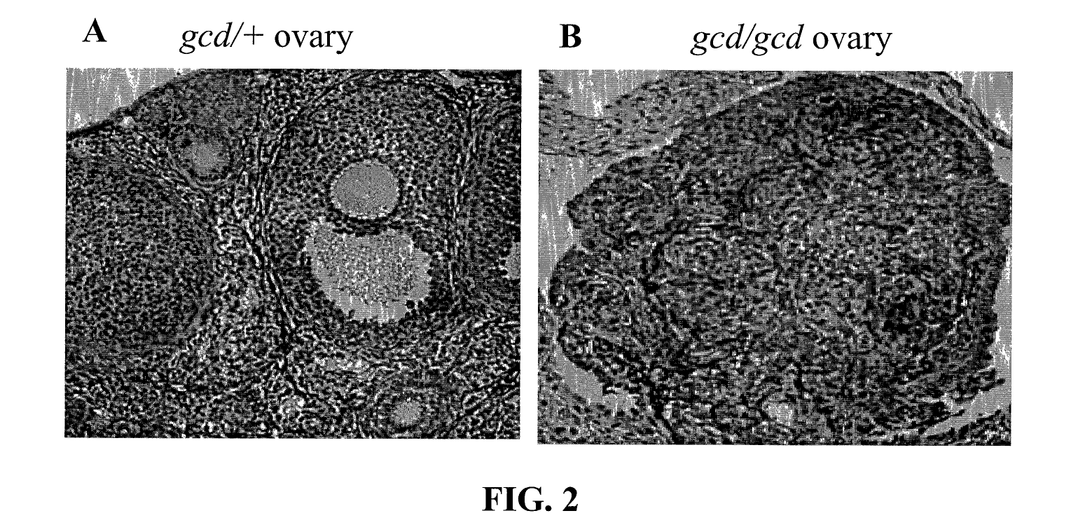

To investigate cellular morphology in gcd/gcd female mutants, which mimic POF in human females, ovaries were extracted from the gcd/gcd mutants or littermate gcd/+ controls, sectioned, and stained with standards reagents known in the art. FIGS. 2A and 2B demonstrate abnormal gcd/gcd ovary tissue compared to littermate gcd/+ controls. In FIG. 2B, the ovary is much smaller, there are no developing follicles, there are few corpora lutea, and ovarian cysts are present.

### Example 3

### Mapping of Transgene Insertion in gcd Mutants

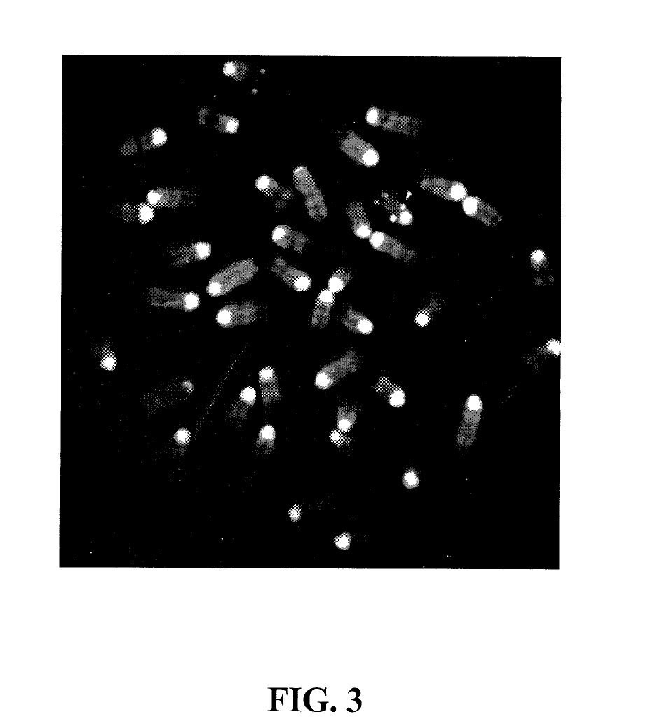

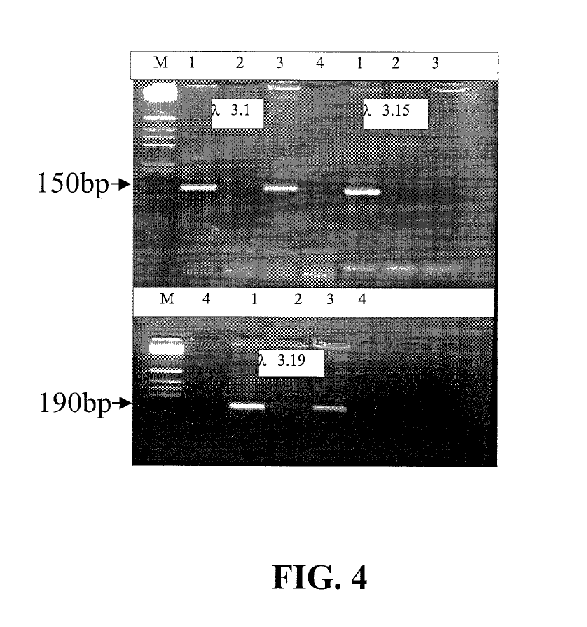

Duncan et al. (1995) had already used standard fluorescent in situ hybridization methods to map the transgene in the gcd mutant to chromosome 11 (FIG. 3). FIG. 4 demonstrates an electrophoretic gel in which PCR reactions are analyzed from the gcd mouse DNA in somatic cell hybrid mapping of the transgene insertion breakpoints in chromosome 11. Primers utilized in mapping experiments include GCD 3.19F

5′-CATGCCTTTCACCTGCTACAC-3′ (SEQ ID NO:11) and GCD 3.19R

5′-GCACTGCTGTCTTTTGAAGCC-3′ (SEQ ID NO:12). FIG. 4 shows polymerization with the given primers in mouse DNA and HM93 DNA (having Rat+Mouse Chromosome 11), but not in controls having rat DNA alone or a water control.

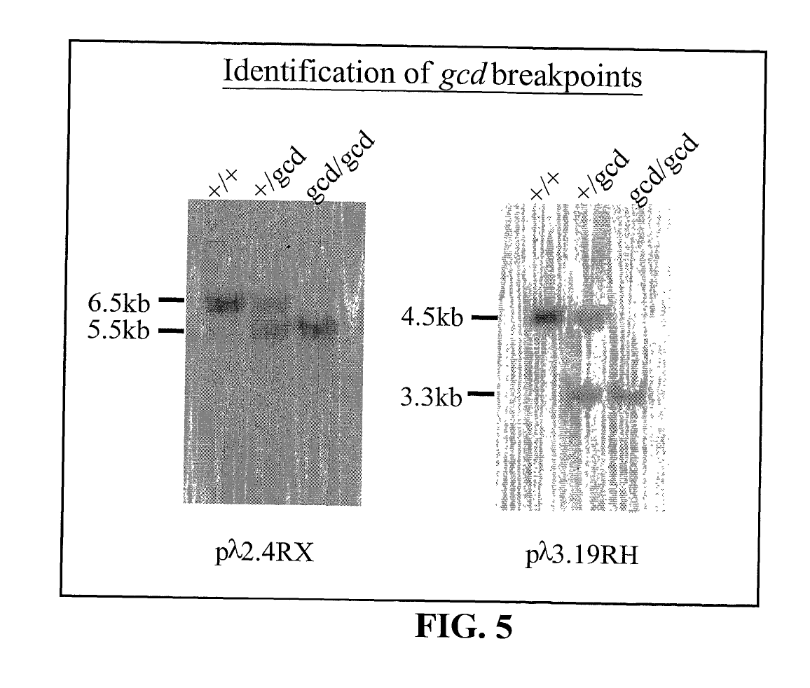

The transgene insertion breakpoints were further analyzed in FIG. 5 in which Southerns, generated by standard methods (see, for example Sambrook et al., 1988) are presented assaying DNA from wild type (+/+), gcd heterozygotes (+/gcd) and gcd mutants (gcd/gcd). Probing the Southern blots with the inserted transgene DNA by standard methods (Sambrook et al., 1988) (pλ2.4RX DNA (left) and pλ3.19RH DNA (right)) identifies the gcd breakpoints.

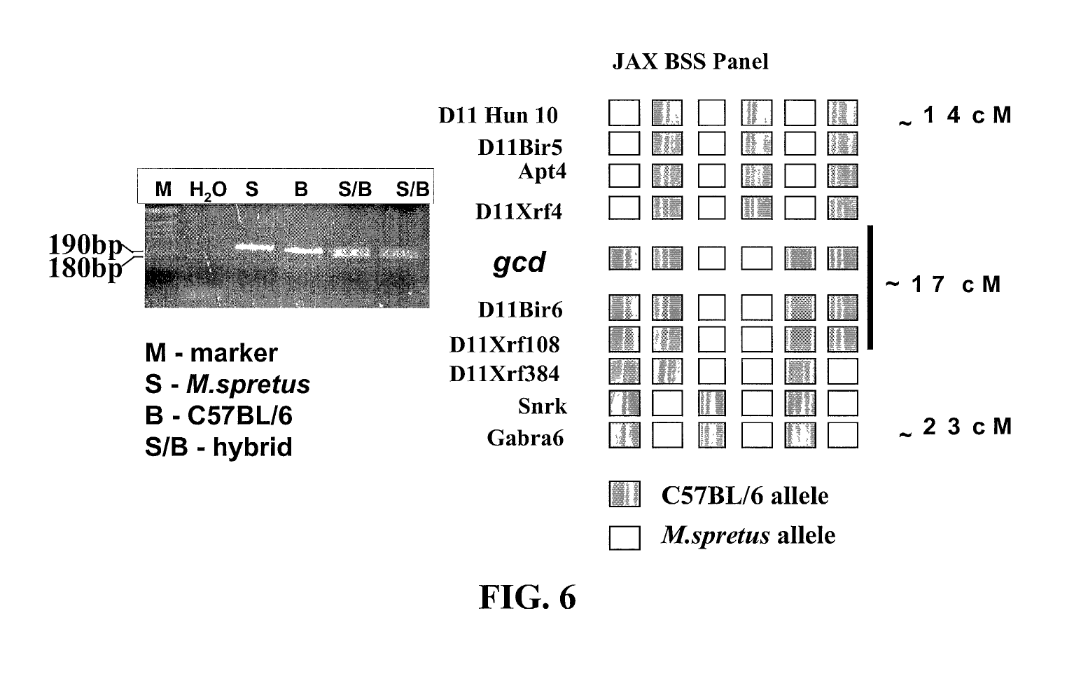

The gcd transgene insertion was further characterized on an interspecies BSS backcross panel (FIG. 6), such as described by Rowe et al., 1994, incorporated by reference herein in its entirety. That is, in order to more accurately map these sequences on chromosome 11 meiotic mapping was employed using the public Jackson Laboratories (Bar Harbor, Me.) M. spretus/M. musculus interspecific backcross mapping panels. The BSS ([B6xSp]xSp) set was chosen as it carried the greatest density of markers in the proximal region of chromosome 11. This panel consists of DNA taken from 94 BSS backcross mice and depending on the region of the genome, allows mapping at a resolution of between 15 cM. In order to detect the required polymorphisms the same λ2.4 and λ3.19 end fragments used above were PCR amplified from M. spretus and C57BL/6 and directly cycle sequenced. No sequence differences between M. spretus and B6 could be found at the λ2.4 locus, but several base pair changes were evident at the λ3.19 locus. One of these changes occurred in a unique SphI site allowing us to use PCR followed by restriction enzyme digestion to reveal this polymorphism. The PCR product is 190 bp for M. spretus and B6. The unique SphI site (GCATGC) is found in B6, 10 bp from one end, whereas the corresponding sequence in M. spretus DNA lacks this recognition site (ACATGC). After PCR amplification and restriction the 190 bp M spretus allele can be distinguished from the 180 bp B6 allele by electrophoresis in a high resolution 4% metaphor agarose gel as shown at left in FIG. 6. Thus, the mutation was mapped to a genetic interval of less than 1 cM using this interspecific backcross analysis, and it was established that the transgene insertion caused a deletion in gcd genomic DNA.

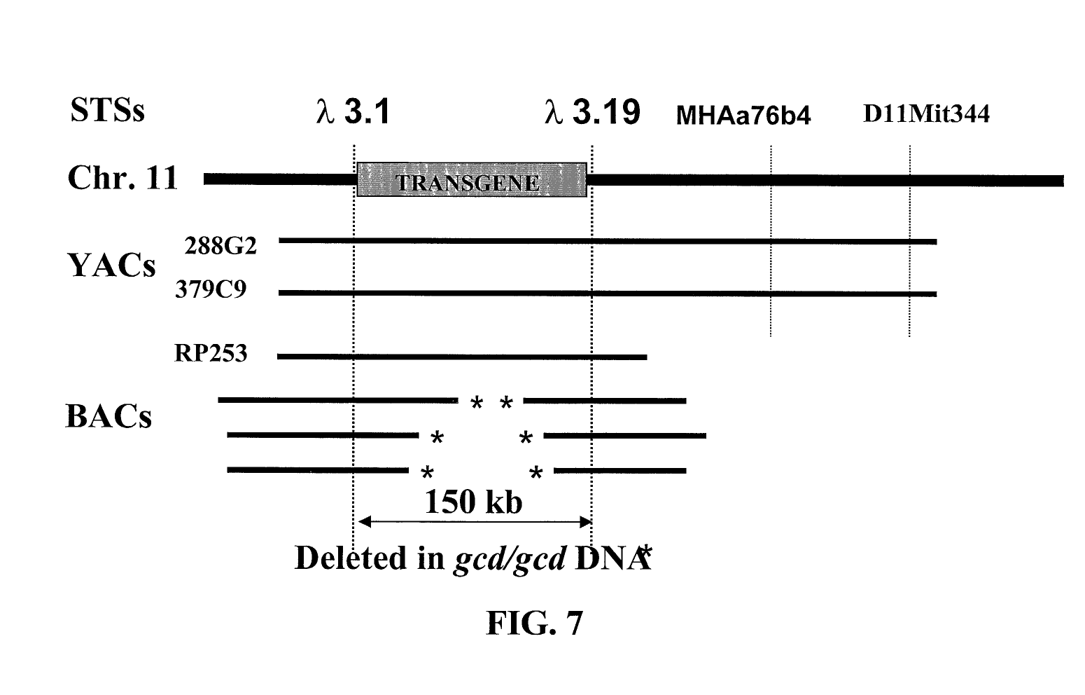

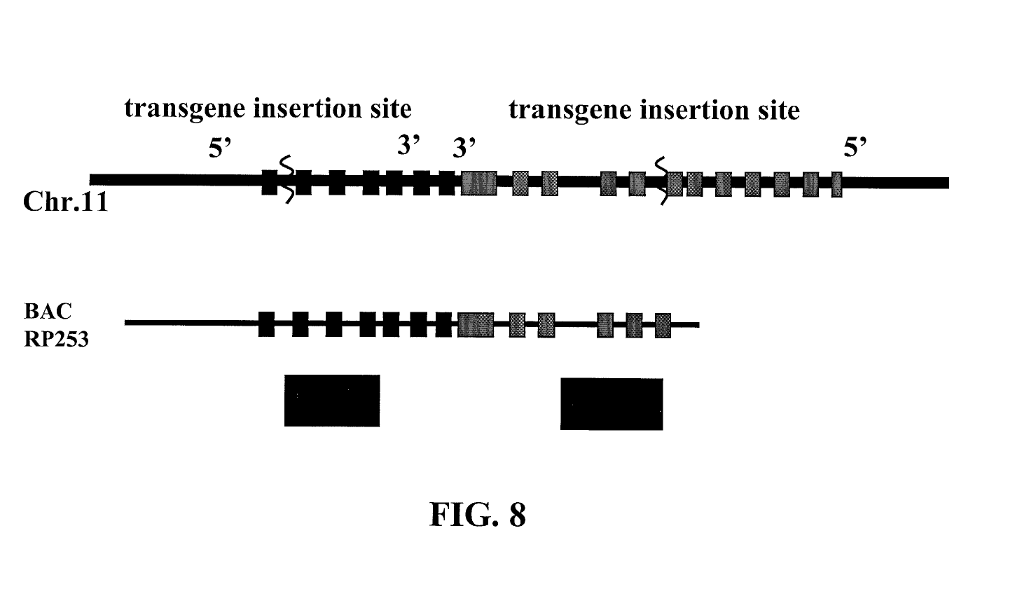

FIG. 7 illustrates that the transgene insertion deletes approximately 150 kb of DNA in the gcd mouse. A BAC contig of the critical region was then constructed. A single 200 kb BAC was identified which fully spans the gcd critical deletion interval. Sample sequencing of the BAC in combination with BLAST analysis of the publicly available mouse and human genomic sequence databases was then performed. Three hundred sixty clones were sequenced by standard methods, comprising approximately 0.9 times coverage. A gene map of the mouse gcd region and also the homologous region of the human (chromosome 2p15-p16) were constructed. Five hits were identified in an EST databank (GenBank), and two novel genes were identified: Vrk2 and Can1 (Candidate 1). Vrk2 (Vaccinia related kinase 2) is a serine/threonine kinase, having widespread expression and being elevated in highly proliferative cells. Can1 has widespread adult expression, and is particularly elevated in testis. FIG. 8 demonstrates the location of the Vrk2 and Can1 genes in the region of chromosome 11 harboring the transgene insertion site. BAC RP253 contains both Vrk2 and Can1 genes. The 3′ exons and UTR regions of both genes were deleted in the gcd mutant.

### Example 4

### VRK2 Does Not Complement gcd Phenotype

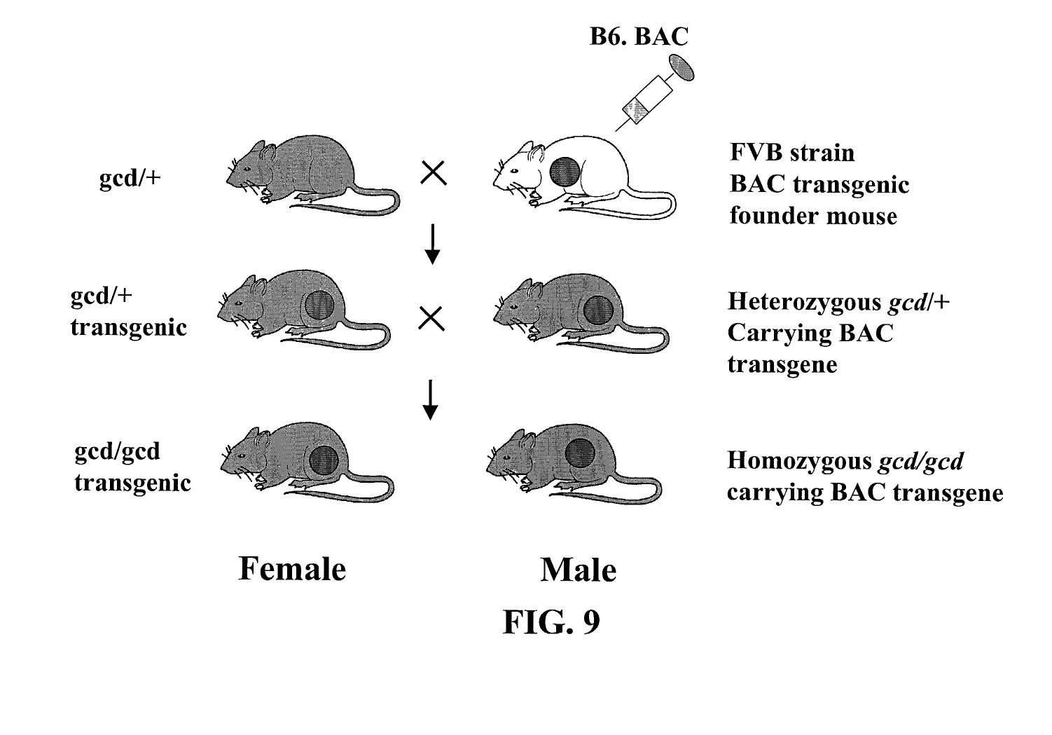

Using transgenic technology methods standard in the art, the Vrk2 gene was tested for the ability to complement the gcd phenotype in gcd mice (FIG. 9). Briefly, gcd/+ females were mated with a FVB strain (Charles River Laboratories; Wilmington, Mass.) BAC transgenic founder mouse transfected with B6.BAC. From the offspring of this cross, transgenic gcd/+ females were mated with heterozygous gcd/+ males carrying the BAC transgene. From the offspring of this cross, gcd/gcd transgenic females and homozygous gcd/gcd males carrying the BAC transgene were analyzed.

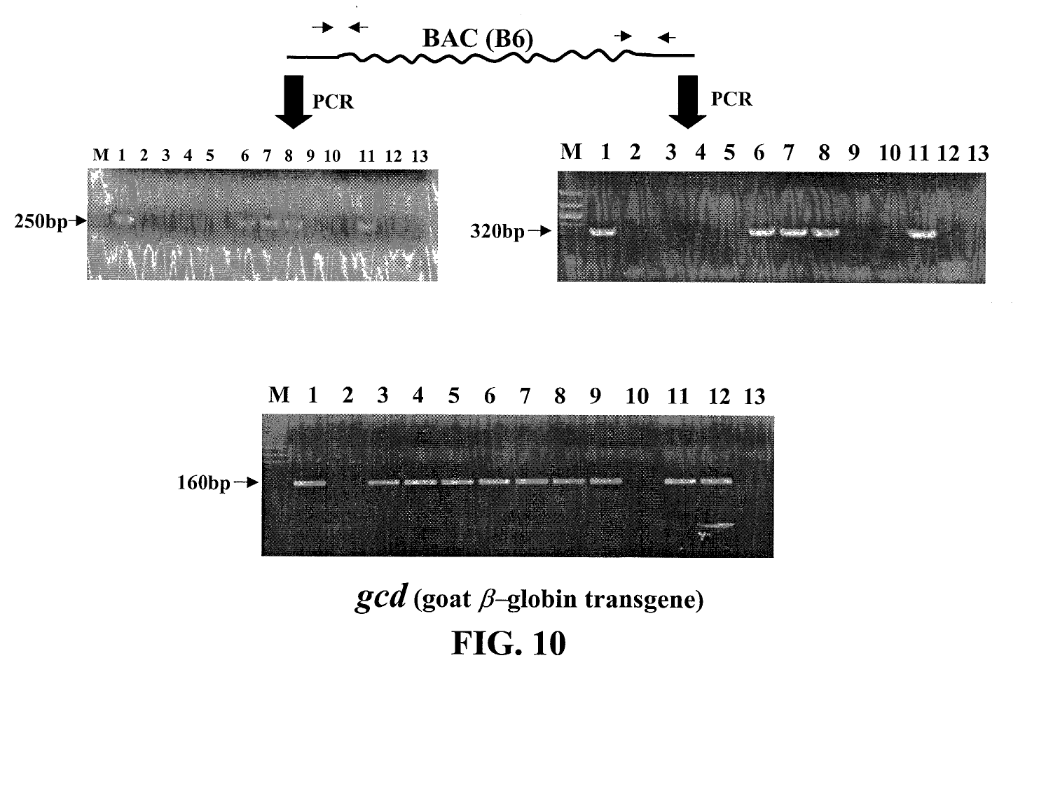

FIG. 10 demonstrates an electrophoresis gel of polymerase chain reaction experiments assaying DNA from 13 different gcd/gcd mice containing the Vrk2 BAC. Top left and right panels show a PCR designed to detect the left and right arms respectively of the Vrk2-containing BAC. It can be seen that only mouse #1,6,7,8,11 contain both ends of the Vrk2-containing BAC. The bottom panel shows the result using primers specific for the goat β-globin transgene (R: 5′-TGGTGTCTGTTTGTGTAGCTG-3′; (SEQ ID NO:13); F: 5′CCTGTGGAACCACACCTTG-3′; (SEQ ID NO:14)). It can be seen that mouse 1,3,4,5,6,7,8,9,11, and 12 carry the transgene.

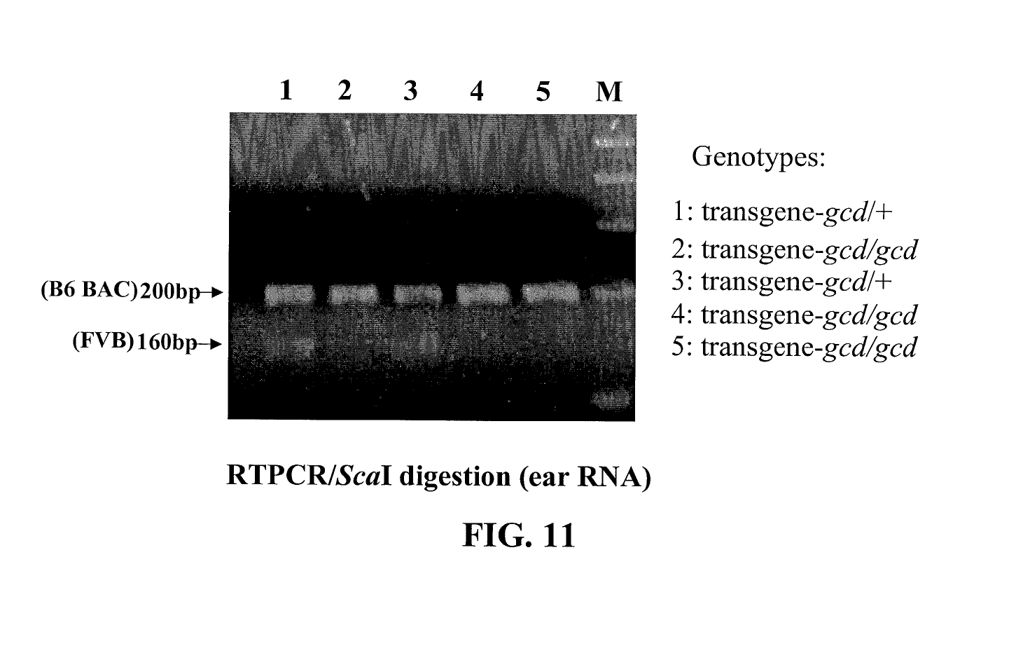

FIG. 11 illustrates identification of gcd/gcd BAC transgenics using a Vrk2 polymorphism. Briefly, tissue from the ears of transgenic mice was harvested and RT/PCR was performed on ear mRNA followed by Scal digestion. Digestion by ScaI denotes a polymorphism associated with Vrk2 DNA. Heterozygous transgenics (gcd/+) (lanes 1 and 3) are identified by a 160 bp fragment absent in homozygous gcd/gcd transgenics (lanes 2, 4 and 5).

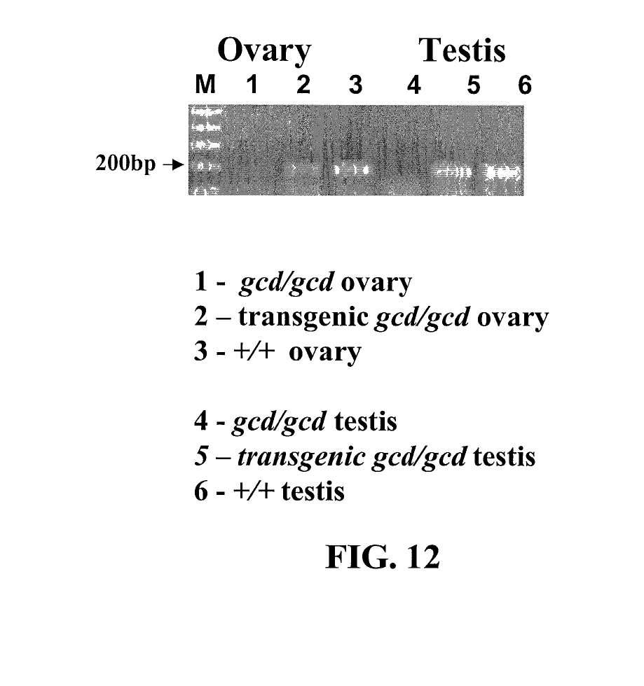

FIG. 12 demonstrates expression of Vrk2 BAC transgene in adult testis and ovaries of gcd/gcd mice. Lanes 1 and 4 are polymerase chain reaction results from gcd/gcd ovary and testis, respectively. Lanes 2 and 5 are from transgenic gcd/gcd ovary and testis, respectively. Lanes 3 and 6 are from wild type (+/+) ovary and testis, respectively. This demonstrates that the Vrk2 gene on the BAC transgene is being expressed in these mice.

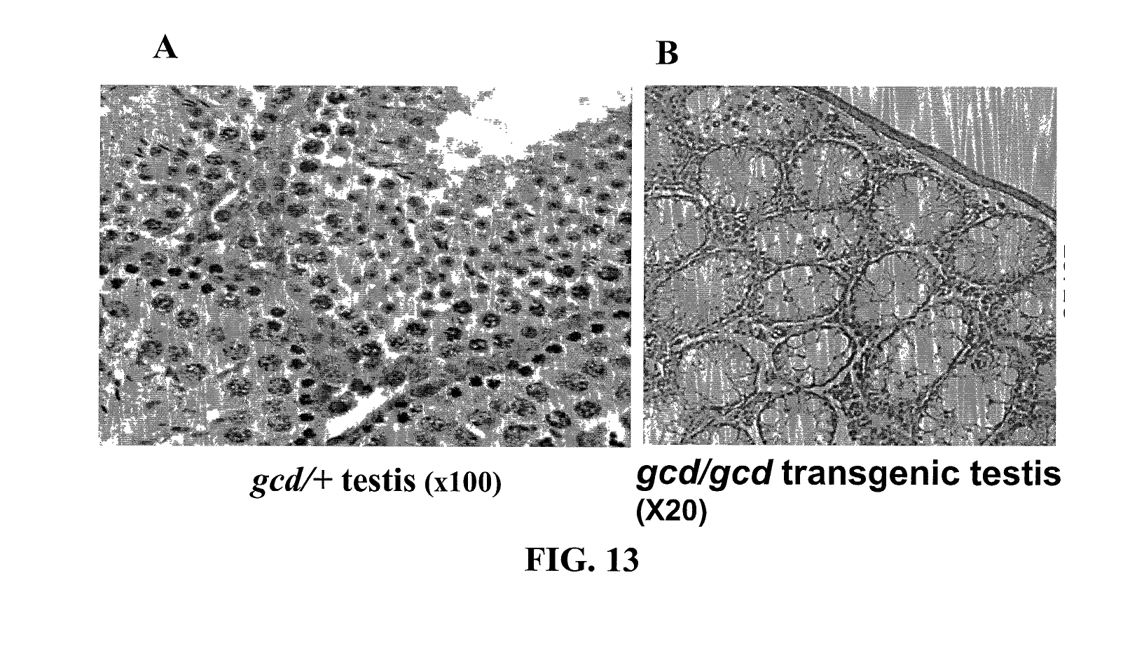

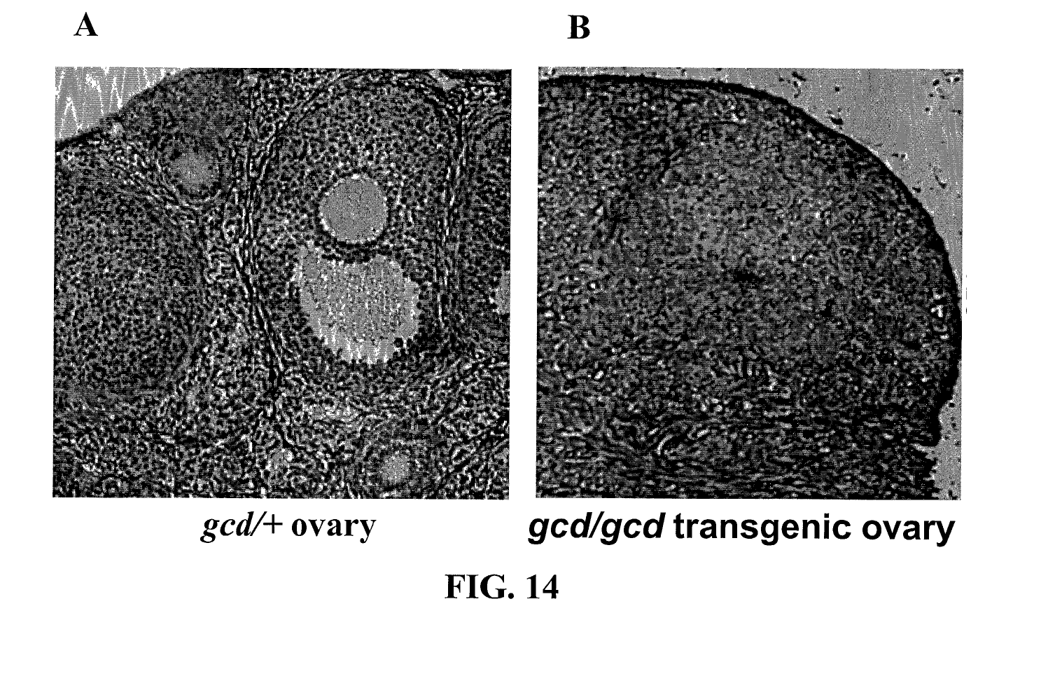

Vrk2 Kinase BAC transgene does not rescue the gcd phenotype in the testis (FIG. 13). Tissue from gcd/+ testis is compared to gcd/gcd transgenic testis which still displays abnormal semeniferous tubules with vacuolated Sertoli cells. The Vrk2 Kinase BAC transgene also does not rescue the gcd phenotype in the adult ovary. Tissue from gcd/+ ovary is compared to gcd/gcd transgenic ovary, which still lacks developing follicles and has few corpora lutea (FIG. 14).

Thus, there was no rescue of the sterility in the Vrk2 BAC-containing transgenics, and the gonadal histology remained typical of the gcd mutant. This indicated that deletion of Vrk2 was not responsible for the gcd phenotype and that Can1 underlies the mutation.

### Example 5

### Can1 is Associated With The gcd Phenotype

The Can1 gene contains 14 exons spread over 100 kb. Expression of the gene produces a 1.7 kb transcript containing a 1.2 kb open reading frame. In a specific embodiment, the gene encodes an intracellular protein. There is high conservation between human and mouse. Northern blots performed by methods standard in the art determined that that there is low level of expression in various adult tissues, although there is significant level of expression particularly in the testis.

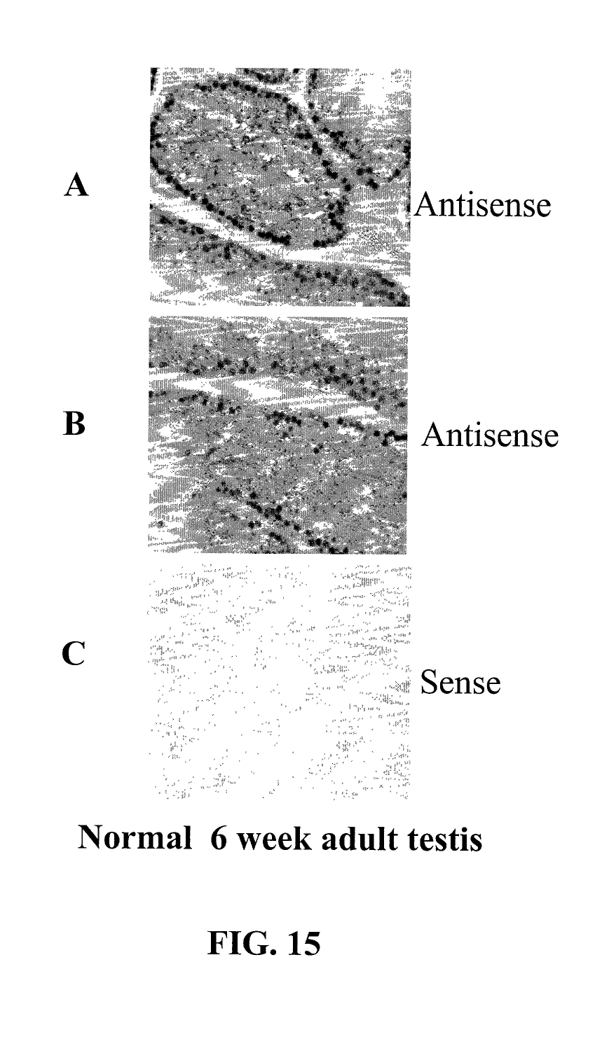

In situ hybridization performed by methods well known in the art shows discreet expression in 12.5 dpc genital ridge, expression is present in normal spermatogonial stem cells (FIG. 15), and there is no detectable expression in normal adult ovaries.

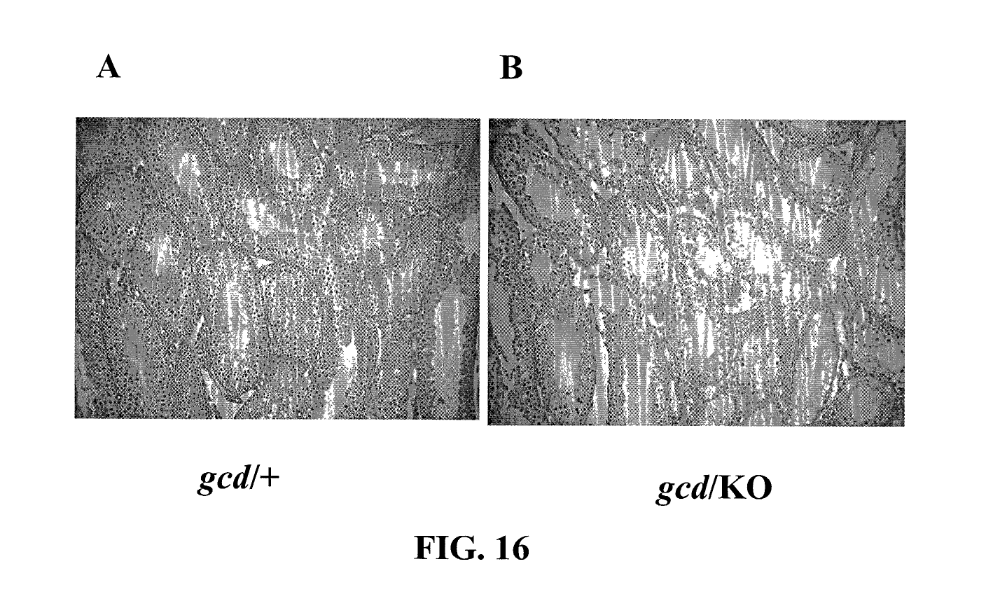

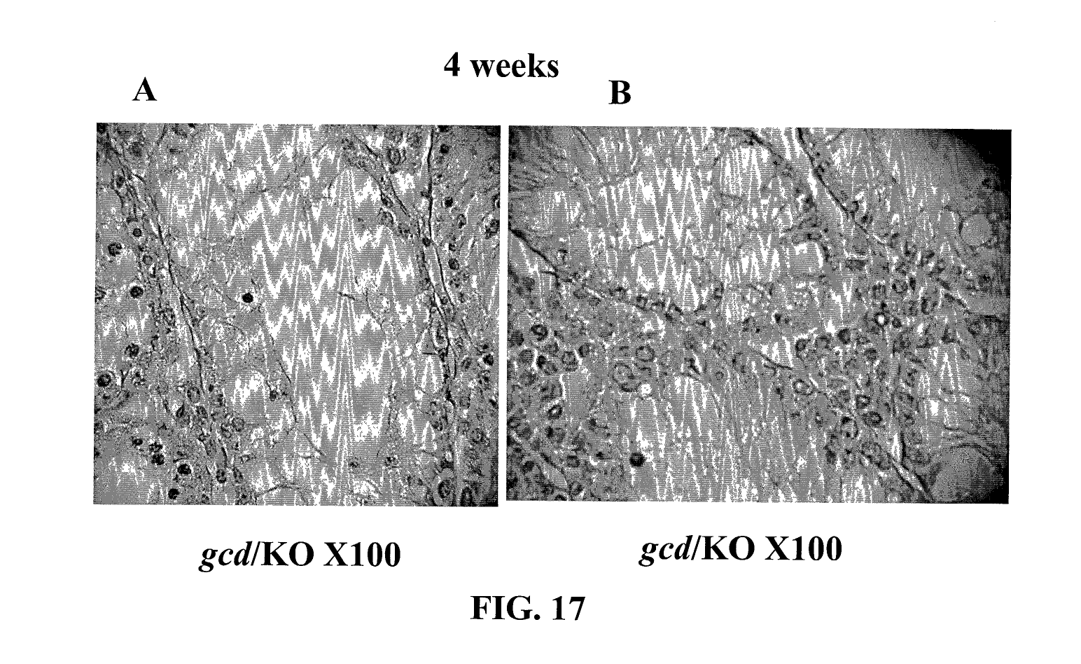

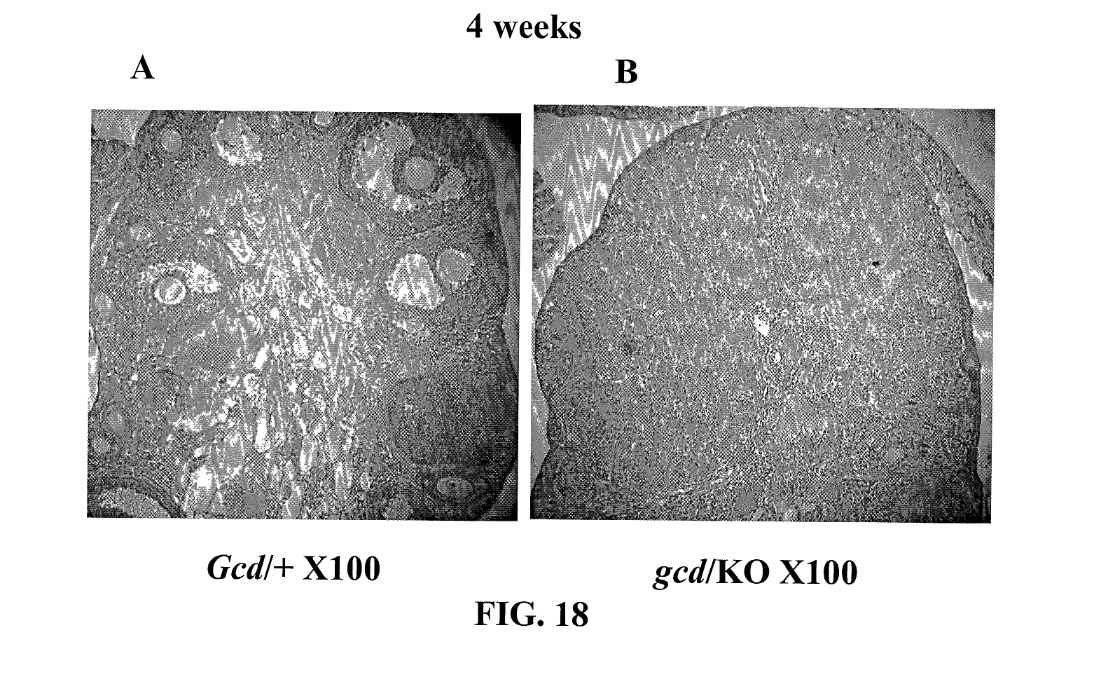

Using gene targeting methods standard in the art, the Can1 gene was disrupted. Ablation of Can1 leads to the typical gcd phenotype of reduced numbers of primordial germ cells, and adult male (FIGS. 16 and 17) and female (FIG. 18) gonads are severely depleted for germ cells.

### Example 6

### Cloning Can1 Human Homologs

The human homologue of Can1 was cloned by standard means in the art. Briefly, a mouse gcd cDNA sequence was used to search a sequence database, such as GenBank maintained at NCBI. A human homolog was identified and obtained by polymerase chain reaction using primers designed corresponding to the human DNA, using well known methods (see Sambrook et al., 1988 and Ausubel et al., 1994, both incorporated by reference herein). In a specific embodiment, the Can1 spatio-temporal expression pattern in embryonic and adult mouse and human is investigated.

### Example 7

### Human Can1 Rescue Experiments in Can1 Knockout Mice

In a specific embodiment, the human Can1 nucleic acid sequence is utilized in a standard transgenic rescue experiment, such as by using Can1 knockout mice, to show that the human Can1 plays a role in fertility.

### Example 8

### Can1 Defects in Infertile Humans

In a specific embodiment, a large number (n=at least about 100) of male SCOS patients and female POF patients can be assessed to determine the clinical impact of Can1 mutations on infertility. Samples are collected from POF- or SCOS-affected individuals and analyzed for the presence of defects in a Can1 nucleic acid and/or Can1 amino acid sequence. Defects in Can1 nucleic acid sequence may by assessed, for example, by such methods as sequencing of part or all of the nucleic acid sequence, polymerase chain reaction, nucleic acid hybridization, restriction enzyme digestion, identification of subcellular localization of the nucleic acid sequence, or a combination thereof. Defects in Can1 amino acid sequence may be assessed, for example, by sequencing of part or all of the amino acid sequence, immunoblot (western) analysis, identification of subcellular localization of the amino acid sequence, or a combination thereof. In a specific embodiment, a significant number of mutations in male SCOS or female POF patients indicates Can1 defects are useful diagnostically or even as a predictor of early reproductive failure. In another specific embodiment, a Can1 protein, polypeptide, or peptide is useful to boost fertility in oligospermic men (low sperm counts) by stimulating germ cell growth.

### Example 9

### Subcellular Localization of Can1

In a specific embodiment, antibodies to Can1 protein, polypeptide or peptide are generated by means standard in the art, and the subcellular localization of the protein product is subsequently determined.

### Example 10

### Yeast Two-Hybrid With Can1 and Other Methods to Identify Can1-Interacting Proteins

In a specific embodiment, a Can1-interacting agent is identified. A skilled artisan recognizes that there are multiple means in the art to identify agents which bind Can1 or a fragment thereof. The agents could be polypeptides, peptides, nucleic acids, small molecules, and the like. Examples of means to identify Can1-interacting agents include two hybrid analysis, affinity binding, coimmunoprecipitation, and such.

In a specific embodiment, yeast two-hybrid analysis is performed by standard means in the art with Can1. The term “two hybrid screen” as used herein refers to a screen to elucidate or characterize the function of a protein by identifying other proteins with which it interacts. The protein of unknown function, herein referred to as the “bait” is produced as a chimeric protein additionally containing the DNA binding domain of GAL4. Plasmids containing nucleotide sequences which express this chimeric protein are transformed into yeast cells, which also contain a representative plasmid from a library containing the GAL4 activation domain fused to different nucleotide sequences encoding different potential target proteins. If the bait protein physically interacts with a target protein, the GAL4 activation domain and GAL4 DNA binding domain are tethered and are thereby able to act conjunctively to promote transcription of a reporter gene. If no interaction occurs between the bait protein and the potential target protein in a particular cell, the GAL4 components remain separate and unable to promote reporter gene transcription on their own. One skilled in the art is aware that different reporter genes can be utilized, including β-galactosidase, HIS3, ADE2, or URA3. Furthermore, multiple reporter sequences, each under the control of a different inducible promoter, can be utilized within the same cell to indicate interaction of the GAL4 components (and thus a specific bait and target protein). A skilled artisan is aware that use of multiple reporter sequences decreases the chances of obtaining false positive candidates. Also, alternative DNA-binding domain/activation domain components may be used, such as LexA. One skilled in the art is aware that any activation domain may be paired with any DNA binding domain so long as they are able to generate transactivation of a reporter gene. Furthermore, a skilled artisan is aware that either of the two components may be of prokaryotic origin, as long as the other component is present and they jointly allow transactivation of the reporter gene, as with the LexA system.

Two hybrid experimental reagents and design are well known to those skilled in the art (see The Yeast Two-Hybrid System by P. L. Bartel and S. Fields (eds.) (Oxford University Press, 1997), including the most updated improvements of the system (Fashena et al., 2000). A skilled artisan is aware of commercially available vectors, such as the Matchmaker™ Systems from Clontech (Palo Alto, Calif.) or the HybriZAP® 2.1 Two Hybrid System (Stratagene; La Jolla, Calif.), or vectors available through the research community (Yang et al., 1995; James et al., 1996). In alternative embodiments, organisms other than yeast are used for two hybrid analysis, such as mammals (Mammalian Two Hybrid Assay Kit from Stratagene (La Jolla, Calif.)) or E. coli (Hu et al., 2000).

In an alternative embodiment, a two hybrid system is utilized wherein proteinprotein interactions are detected in a cytoplasmic-based assay. In this embodiment, proteins are expressed in the cytoplasm, which allows posttranslational modifications to occur and permits transcriptional activators and inhibitors to be used as bait in the screen. An example of such a system is the CytoTrap® Two-Hybrid System from Stratagene™ (La Jolla, Calif.), in which a target protein becomes anchored to a cell membrane of a yeast which contains a temperature sensitive mutation in the cdc25 gene, the yeast homolog for hSos (a guanyl nucleotide exchange factor). Upon binding of a bait protein to the target, hSos is localized to the membrane, which allos activation of RAS by promoting GDP/GTP exchange. RAS then activates a signaling cascade which allows growth at 37° C. of a mutant yeast cdc25H. Vectors (such as pMyr and pSos) and other experimental details are available for this system to a skilled artisan through Stratagene (La Jolla, Calif.). (See also, for example, U.S. Pat. No. 5,776,689, herein incorporated by reference).

Thus, in accordance with an embodiment of the present invention, there is a method of screening for a peptide which interacts with Can1 comprising introducing into a cell a first nucleic acid comprising a DNA segment encoding a test peptide, wherein the test peptide is fused to a DNA binding domain, and a second nucleic acid comprising a DNA segment encoding at least part of Can1, respectively, wherein the at least part of Can1, respectively, is fused to a DNA activation domain. Subsequently, there is an assay for interaction between the test peptide and the Can1 polypeptide or fragment thereof by assaying for interaction between the DNA binding domain and the DNA activation domain. In a preferred embodiment, the assay for interaction between the DNA binding and activation domains is activation of expression of β-galactosidase.

### Example 11

### Chip Expression Technology with Can1

In another specific embodiment, chip expression technology is performed to determine other nucleic acids which are either upregulated or downregulated by Can1.

### Example 12

### Identification of Functional Domains

In a specific embodiment, Can1 functional domains are determined by methods standard in the art. For example, Can1 sequences from different organisms are compared to identify regions which are conserved between the species. In an alternative embodiment, regions of the Can1 amino acid sequence are utilized in two hybrid vectors, and it is determined which region is required for interaction with a target protein or polypeptide. Other methods are well known in the art.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

### PATENTS

U.S. Pat. No. 4,578,770, issued Mar. 25, 1986

U.S. Pat. No. 4,596,792, issued Jun. 24, 1986.

U.S. Pat. No. 4,599,230, issued Jul. 8, 1986.

U.S. Pat. No. 4,599,231, issued Jul. 8, 1986.

U.S. Pat. No. 4,601,903, issued Jul. 22, 1986.

U.S. Pat. No. 4,608,251, issued Aug. 26, 1986.

U.S. Pat. No. 5,840,873, issued Nov. 24, 1998

U.S. Pat. No. 5,843,640, issued Dec. 1, 1998

U.S. Pat. No. 5,843,650, issued Dec. 1, 1998

U.S. Pat. No. 5,843,651, issued Dec. 1, 1998

U.S. Pat. No. 5,843,663, issued Dec. 1, 1998

U.S. Pat. No. 5,846,708, issued Dec. 8, 1998

U.S. Pat. No. 5,846,709, issued Dec. 8, 1998

U.S. Pat. No. 5,846,717, issued Dec. 8, 1998

U.S. Pat. No. 5,846,726, issued Dec. 8, 1998

U.S. Pat. No. 5,846,729, issued Dec. 8, 1998

U.S. Pat. No. 5,846,783, issued Dec. 8, 1998

U.S. Pat. No. 5,849,481, issued Dec. 15, 1998

U.S. Pat. No. 5,849,483, issued Dec. 15, 1998

U.S. Pat. No. 5,849,486, issued Dec. 15, 1998

U.S. Pat. No. 5,849,487, issued Dec. 15, 1998

U.S. Pat. No. 5,849,497, issued Dec. 15, 1998

U.S. Pat. No. 5,849,546, issued Dec. 15, 1998

U.S. Pat. No. 5,849,547, issued Dec. 15, 1998

U.S. Pat. No. 5,851,770, issued Dec. 22, 1998

U.S. Pat. No. 5,851,772, issued Dec. 22, 1988

U.S. Pat. No. 5,853,990, issued Dec. 29, 1998

U.S. Pat. No. 5,853,993, issued Dec. 29, 1998

U.S. Pat. No. 5,853,992, issued Dec. 29, 1998

U.S. Pat. No. 5,856,092, issued Jan. 5, 1999

U.S. Pat. No. 5,858,652, issued Jan. 12, 1999

U.S. Pat. No. 5,861,244, issued Jan. 19, 1999

U.S. Pat. No. 5,863,732, issued Jan. 26, 1999

U.S. Pat. No. 5,863,753, issued Jan. 26, 1999

U.S. Pat. No. 5,866,331, issued Feb. 2, 1999

U.S. Pat. No. 5,866,336, issued Feb. 2, 1999

U.S. Pat. No. 5,866,337, issued Feb. 2, 1999

U.S. Pat. No. 5,900,481, issued May 4, 1999

U.S. Pat. No. 5,905,024, issued May 18, 1999

U.S. Pat. No. 5,910,407, issued Jun. 8, 1999

U.S. Pat. No. 5,912,124, issued Jun. 5, 1999

U.S. Pat. No. 5,912,145, issued Jun. 15, 1999

U.S. Pat. No. 5,912,148, issued Jun. 15,1999

U.S. Pat. No. 5,916,776, issued Jun. 29, 1999

U.S. Pat. No. 5,916,779, issued Jun. 29, 1999

U.S. Pat. No. 5,919,626, issued Jul. 6, 1999

U.S. Pat. No. 5,919,630, issued Jul. 6, 1999

U.S. Pat. No. 5,922,574, issued Jul. 13, 1999

U.S. Pat. No. 5,925,517, issued Jul. 20, 1999

U.S. Pat. No. 5,925,525, issued Jul. 20, 1999

U.S. Pat. No. 5,928,862, issued Jul. 27, 1999

U.S. Pat. No. 5,928,869, issued Jul. 27, 1999

U.S. Pat. No. 5,928,870, issued, Jul. 27, 1999

U.S. Pat. No. 5,928,905, issued Jul. 27, 1999

U.S. Pat. No. 5,928,906, issued Jul. 27, 1999

U.S. Pat. No. 5,929,227, issued Jul. 27, 1999

U.S. Pat. No. 5,932,413, issued Aug. 3, 1999

U.S. Pat. No. 5,932,451, issued Aug. 3, 1999

U.S. Pat. No. 5,935,791, issued Aug. 10, 1999

U.S. Pat. No. 5,935,825, issued Aug. 10, 1999

U.S. Pat. No. 5,939,291, issued Aug. 17, 1999

U.S. Pat. No. 5,942,391, issued Aug. 24, 1999

European Application No. 320308

European Application No. 329 822

GB Application No. 2 202 328

PCT Application No. PCT/US87/00880

PCT Application No. PCT/US89/01025

PCT Application WO 88/10315

PCT Application WO 89/06700

PCT Application WO 90/07641

### PUBLICATIONS

Bangham et al, J. Mol. Biol., 13:238, 1965.

Deamer and Uster, “Liposome Preparation: Methods and Mechanisms,” LIPOSOMES, M. Ostro ed. (1983).

Duncan, M K and Chaka, K K. Incidence of tubulostromal adenoma of the ovary in aged germ cell-deficient mice. (1993) J. Comp. Path. 109: 13-19.

Duncan, M, Cummings, L, and Chada, K. Germ cell deficient (gcd) mouse as a model of premature ovarian failure. (1993). Biology of Reproduction 49: 221-227.

Duncan, M K, Lieman, J, and Chada, K K. The germ cell deficient locus maps to mouse chromosome 11A2-3. (1995). Mammalian Genome 6:697-699.

Ghosh and Bachhawat, “Targeting of liposomes to hepatocytes,” In: Wu G. Wu C ed., Liver diseases, targeted diagnosis and therapy using specific receptors and ligands, New York: Marel Dekker, pp. 87-104, 1991.

Gregoriadis, DRUG CARRIERS IN BIOLOGY AND MEDICINE, G. Gregoriadis (ed.), 1979, pp. 287-341.

Kaneda et al., “Increased expression of DNA cointroduced with nuclear protein in adult rat liver,” Science, 243:375-378, 1989.

Kato et al., “Expression of hepatitis B virus surface antigen in adult rat liver,” J. Biol. Chem., 266:3361-3364, 1991.

Leon, O and Roth, M, Zinc fingers: DNA binding and protein-protein interactions, Biol Res 33: 21-30, 2000.

Nicolau et al., “Liposomes as carriers for in vivo gene transfer and expression,” Methods Enzymol., 149:157-176, 1987.

Nistal, M, Jimenez, F, and Paniagua R, Sertoli cell types in the Sertoli-cell-only syndrome:

relationships between Sertoli cell morphology and aetiology. (1990) Histopathology 16: 173-180.

Pellas, T C, Ramachandran, B, Duncan, M, Pan, S S, Marone, M, and Chada, K. Germ-cell deficient (gcd), an insertional mutation manifested as infertility in transgenic mice. (1991). Proc. Natl. Acad. Sci. USA 88: 8787-8791.

Remington's Pharmaceutical Sciences 15th Edition, pages 1035-1038, 1570-1580.

Rowe L B, Nadeau J H, Turner R, Frankel W N, Letts V A, Eppig J T, Ko M S, Thurston S J, Birkenmeier E H. Maps from two interspecific backcross DNA panels available as a community genetic mapping resource. Mamm Genome. 1994 May;5(5):253-74.

Sambrook, Fritsch, Maniatis, In: Molecular Cloning: A Laboratory Manual, Vol. 1, Cold Spring Harbor Laboratory Press, Cold Spring Harbor, N.Y., Ch. 7,7.19-17.29, 1989.

Saurin, A J, Borden, K L, Boddy, M N, Freemont, P S (1996) Does this have a familiar RING? Trends Biochem Sci 21: 208-214.

Szoka and Papahadjopoulos, Proc. Nat'l Acad. Sci. U.S.A. 75:4194-98 (1978).

Wong et al., “Appearance of β-lactamase activity in animal cells upon liposome mediated gene transfer,” Gene, 10:87-94, 1980.

One skilled in the art readily appreciates that the patent invention is well adapted to carry out the objectives and obtain the ends and advantages mentioned as well as those inherent therein. Can1 sequences, pharmaceutical compositions, methods, treatments, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

We claim:

1. As a composition of matter, a nucleic acid sequence of SEQ ID NO:1.

2. As a composition of matter, an amino acid sequence of SEQ ID NO:3.

3. A pharmaceutical composition comprising:
an amino acid sequence of SEQ ID NO:3; and
a pharmacologically acceptable carrier.

4. A method of treating infertility in a mammal, comprising the step of introducing a therapeutically effective amount of a Can1 nucleic acid sequence into said mammal.

5. A method of treating infertility in a mammal, comprising the step of introducing a therapeutically effective amount of a nucleic acid sequence of SEQ ID NO:1 into said mammal.

6. The method of claim 4 or 5, wherein said nucleic acid sequence is introduced into said mammal in a vector.

7. The method of claim 6, wherein said vector is selected from the group consisting of a plasmid, an adenovirus vector, an adeno-associated viral vector, a retroviral vector, a liposome, and a combination thereof.

8. A method of treating infertility in a mammal, comprising the step of introducing a therapeutically effective amount of a nucleic acid sequence of SEQ ID NO:2 into said mammal.

9. The method of claim 8, wherein said nucleic acid sequence is introduced into said mammal in a vector.

10. The method of claim 9, wherein said vector is selected from the group consisting of a plasmid, an adenovirus vector, an adeno-associated viral vector, a retroviral vector, a liposome, and a combination thereof.

11. A method of treating infertility in a mammal comprising the step of introducing into said mammal a therapeutically effective amount of an amino acid sequence of SEQ ID NO:3.

12. A method of treating infertility in a mammal comprising the step of introducing to said mammal a therapeutically effective amount of an amino acid sequence of SEQ ID NO:4.

13. The method of claim 11 or 12, wherein said amino acid sequence further comprises a protein transduction domain.

14. A method of diagnosing infertility in a mammal, comprising the steps of:
obtaining a biological sample from said mammal, wherein said sample includes a Can1 nucleic acid sequence; and
assaying for a defect in said Can1 nucleic acid sequence.

15. The method of claim 14, wherein said assaying step comprises an assay selected from the group consisting of polymerase chain reaction, nucleic acid hybridization, DNA chip analysis, sequencing, electrophoresis, and a combination thereof.

16. A method of diagnosing infertility in a mammal, comprising the steps of:
obtaining a biological sample from said mammal, wherein said sample includes a Can1 amino acid sequence; and
assaying for a defect in said Can1 amino acid sequence.

17. The method of claim 16, wherein said assaying step comprises an assay selected from the group consisting of mass spectrometry, sequencing, electrophoresis, immunoblot analysis, subcellular localization and a combination thereof.

18. A method of increasing the number of primordial germ cells in a mammal, comprising the step of introducing into said mammal a physiologically significant level of a Can1 nucleic acid sequence.

19. A method of increasing the number of primordial germ cells in a mammal, comprising the step of introducing into said mammal a physiologically significant level of a Can1 amino acid sequence.

20. A method of stimulating germ cell growth in a mammal, comprising the step of introducing into said mammal a physiologically effective level of a Can1 nucleic acid sequence.

21. A method of stimulating germ cell growth in a mammal, comprising the step of introducing into said mammal a physiologically effective level of a Can1 amino acid sequence.

22. A method of screening for an active compound for the treatment of infertility, comprising the steps of:
obtaining an organism, wherein the genome of said organism includes a reporter sequence whose expression is controlled by a Can1 regulatory nucleic acid sequence;
exposing a test agent to said organism; and
measuring a change in said expression, wherein said change indicates said test agent is said active compound.

23. The method of claim 22, wherein said organism is a mouse.

24. The method of claim 22, wherein said change in expression is an increase in expression.

25. A method of screening in vitro for an active compound for the treatment of infertility, comprising the steps of:
obtaining a cell, wherein said cell includes a nucleic acid sequence having a reporter sequence and wherein the expression of said reporter sequence is controlled by a Can1 regulatory nucleic acid sequence;
exposing a test agent to said cell; and
measuring a change in said expression, wherein said change indicates said test agent is said active compound.

26. The method of claim 25, wherein said cell is a mouse cell.

27. The method of claim 25, wherein said reporter sequence is selected from the group consisting of β-galactosidase, β-glucuronidase, green fluorescent protein, blue fluorescent protein, and chloramphenicol acetyltransferase.

28. The method of claim 25, wherein said change in said expression is an increase in said expression.

29. A method of screening for a candidate substance for the treatment of infertility comprising the steps of:
providing a cell lacking a functional Can1 amino acid sequence;
contacting said cell with said candidate substance; and
determining the effect of said candidate substance on said cell, wherein said effect on said cell is indicative said candidate substance treats infertility.

30. A transgenic non-human animal whose genome comprises a transgene encoding a Can1 amino acid sequence, wherein said transgene is under the control of an operably linked promoter active in eukaryotic cells.

31. The animal of claim 30, wherein said promoter is constitutive.

32. The animal of claim 30, wherein said promoter is tissue specific.

33. The animal of claim 30, wherein said promoter is inducible.

34. The animal of claim 30, wherein said animal is a mouse.

35. A transgenic non-human animal comprising a genome with a heterozygous disruption of a nucleic acid encoding a Can1 polypeptide.

36. A transgenic non-human animal comprising a genome with a homozygous disruption of a nucleic acid encoding a Can1 polypeptide.

37. A method of identifying an upregulator of Can1 nucleic acid sequence expression comprising the steps of:
administering a test compound to an animal of claim 30;
measuring the level of said Can1 expression; and
comparing the level of said Can1 expression in said animal with normal Can1 expression, wherein an increase in said level following administration of said test compound indicates said test compound is an upregulator.

38. A monoclonal antibody that binds immunologically to a polypeptide comprising SEQ ID NO:3, or an antigenic fragment thereof.

39. A polyclonal antisera, antibodies of which bind immunologically to a polypeptide comprising SEQ ID NO:3, or an antigenic fragment thereof.

40. A monoclonal antibody that binds immunologically to a polypeptide comprising SEQ ID NO:4, or an antigenic fragment thereof.

41. A polyclonal antisera, antibodies of which bind immunologically to a polypeptide comprising SEQ ID NO:4, or an antigenic fragment thereof.

42. A method of screening for an active compound for infertility, comprising the steps of:
introducing into a cell
a first nucleic acid expressing a fused test peptide/DNA binding domain; and
a second nucleic acid expressing a fused Can1 polypeptide/DNA activation domain; and
assaying for an interaction between said test peptide and said Can1 polypeptide by measuring binding between said DNA binding domain and said DNA activation domain, wherein said interaction between said test peptide and said Can1 polypeptide indicates said test peptide is said active compound.

43. A method of treating an individual for premature ovarian failure, comprising the step of administering to said individual a nucleic acid sequence of SEQ ID NO:1.

44. A method of treating an individual for premature ovarian failure, comprising the step of administering to said individual a nucleic acid sequence of SEQ ID NO:2.

45. A method of treating an individual for premature ovarian failure, comprising the step of administering to said individual an amino acid sequence of SEQ ID NO:3.

46. A method of treating an individual for premature ovarian failure, comprising the step of administering to said individual an amino acid sequence of SEQ ID NO:4.

47. A method of treating an individual for Sertoli Cell only syndrome, comprising the step of administering to said individual a nucleic acid sequence of SEQ ID NO:1.

48. A method of treating an individual for Sertoli Cell only syndrome, comprising the step of administering to said individual a nucleic acid sequence of SEQ ID NO:2.

49. A method of treating an individual for Sertoli Cell only syndrome, comprising the step of administering to said individual an amino acid sequence of SEQ ID NO:3.

50. A method of treating an individual for Sertoli Cell only syndrome, comprising the step of administering to said individual an amino acid sequence of SEQ ID NO:4.
